(19) United States
(12) Patent Application Publication
(10) Pub. No.: US 2026/0210975 A1
(43) Pub. Date: Jul. 23, 2026

(54) DIAGNOSIS AND TREATMENT OF LONG-COVID

(71) Applicant: LONDON HEALTH SCIENCES CENTRE RESEARCH INC., London (CA)

(72) Inventors: Douglas Fraser, London (CA); Cristiana Iosef, London (CA)

(73) Assignee: LONDON HEALTH SCIENCES CENTRE RESEARCH INC., London, ON (CA)

(21) Appl. No.: 19/137,223

(22) PCT Filed: Dec. 15, 2023
(86) PCT No.: PCT/CA2023/051680

§ 371 Date: Jun. 9, 2025

Related U.S. Application Data
(60) Provisional application No. 63/433,425, filed on Dec. 16, 2022.

(51) Int. Cl. G01N 33/68; A61K 31/16; A61K 31/27; A61K 31/445; A61K 31/55; C07K 16/18; G16H 50/20

(52) U.S. Cl. CPC G01N 33/6896; A61K 31/16; A61K 31/27; A61K 31/445; A61K 31/55; C07K 16/18; G16H 50/20; G01N 2800/2821; G01N 2800/50; G01N 2800/52

(57) ABSTRACT

A method of determining a risk of developing a neurological disorder in a Long-COVID patient comprising: (a) testing levels of at least one marker associated with a neurologic disorder in a sample taken from the Long-COVID patient, and (b) making a determination that the Long-COVID patient is at risk of developing said neurological disorder when the levels of said at least one marker is increased in the Long-COVID patient compared to healthy control reference levels of said marker. Also a method of determining a risk of developing a cardiometabolic injury in a Long-COVID patient when the levels of expression of a marker associated to a cardiometabolic injury is different in the Long-COVID patient than the levels of said marker in a healthy control. Also methods of treating Long-COVID with a drug effective to mediate the HIF signaling pathway.

### FIELD

This disclosure relates to Long-COVID, more particular to the diagnosis and treatment of Long-COVID and to organ-specific biomarkers of Long-COVID.

### BACKGROUND

The “Long-COVID” syndrome is also referred to as long-haul COVID, post-COVID-19 condition, chronic COVID and post-acute sequelae of SARS-COV-2 (PASC) [1]. Symptoms of Long-COVID can be either similar or dissimilar from those of acute COVID-19. While some patients have symptoms that last for weeks, months or years after the initial diagnosis [2,3] some recovered from COVID-19 and then endure a return of the symptoms, or they acquire new symptoms [4-7]. Additionally, individuals who had no symptoms when they were infected can still develop symptoms at a later date [1,2]. Mostly, a spectrum of COVID-19 severities drive Long-COVID symptoms [1-3].

Long-COVID investigations are hampered by a variety of symptoms reported, as well as other contributing factors such as i) biological sex, ii) older age, iii) severity of initial COVID-19 illness, iv) amplitude of the immune response to initial infection, v) vaccination status, vi) the SARS-COV-2 variant that caused the initial infection, vii) the severity of any preexisting health conditions (diabetes, lung problems, autoimmune diseases, or obesity), and/or viii) health care inequities [1, 2, 4-7]. A number of mechanisms have been proposed for Long-COVID, including i) reactivated SARS-COV-2 particles [8,9], ii) epigenetically programmed, overactive immune cells that release inflammatory substances [1], iii) autoimmune disease triggered by SARS-COV-2 infection [10-13], or iv) a combination of the above factors originating from the initial COVID-19 disease [5, 10-19]. We recently reported that a panel of vascular transformation biomarkers was significantly elevated in plasma from Long-COVID outpatients (e.g., ANGPT1, MMP1, VEGF-A, and other biomarkers), suggesting that altered angiogenesis may be a common mechanism in these patients with prolonged, diffuse, diverse symptoms [4, 5].

Organ specific prognostic biomarkers are lacking, making the identification of biomarkers and pathophysiological mechanisms critical to optimize Long-COVID care.

### SUMMARY

In one embodiment, the present disclosure relates to a method of determining a risk of developing a neurological disorder in a Long-COVID patient comprising: (a) testing/measuring levels of at least one marker associated with a neurologic disorder in a sample taken from the Long-COVID patient, and (b) making a determination that the Long-COVID patient is at risk of developing said neurological disorder when the levels of said at least one marker is increased in the sample taken from the Long-COVID patient compared to healthy control reference levels of said at least one marker.

In one embodiment of the method of determining a risk of developing a neurological disorder in a Long-COVID patient of the present disclosure, the at least one marker associated with the neurologic disorder includes KIAA0319, S100A14 and SNAPIN.

In another embodiment of the method of determining a risk of developing a neurological disorder in a Long-COVID patient of the present disclosure, the determination is made using machine learning.

In another embodiment of the method of determining a risk of developing a neurological disorder in a Long-COVID patient of the present disclosure, the method further comprises treating the Long-COVID patient with a drug or agent effective to prevent or treat the neurological disorder.

In another embodiment, the present disclosure provides for a method of determining a risk of developing a cardiometabolic injury in a Long-COVID patient comprising (a) testing/measuring the levels of at least one marker associated with a cardiometabolic injury in a sample taken from the Long-COVID patient, and (b) making a determination that the Long-COVID patient is at risk of developing said cardiometabolic injury when the levels of said at least one marker in the sample taken from the Long-COVID patient is different to healthy control reference levels of said at least one marker.

In one embodiment of the method of determining a risk of developing a cardiometabolic injury in a Long-COVID patient of the present disclosure, the at least one marker associated with the cardiometabolic injury are both FN1 and CCL5, and the determination is made when the level of FN1 goes down and the level of CCL5 goes up in the sample taken from the Long-COVID patient relative to healthy control reference level of FN1 and healthy control reference level of CCL5.

In another embodiment of the method of determining a risk of developing a cardiometabolic injury in a Long-COVID patient of the present disclosure, the at least one marker associated with the cardiometabolic injury includes FN1, CCL5, MMP7, HEBP1, MNDA and GPNMB.

In another embodiment of the method of determining a risk of developing a cardiometabolic injury in a Long-COVID patient of the present disclosure, the determination is made using machine learning.

In another embodiment of the method of determining a risk of developing a cardiometabolic injury in a Long-COVID patient of the present disclosure, the method further comprises treating the Long-COVID patient with a drug effective to treat the cardiometabolic injury.

In another embodiment, the present disclosure relates to a method of diagnosing Long-COVID in a patient, the method comprising comparing the level of at least one marker in a sample taken from the patient to a control level of said at least one marker in a sample of any one of a healthy control subject, a mild COVID-19 control subject and a severe COVID-19 control subject, wherein the patient is diagnosed as having Long-COVID when the level of said at least one marker is increased in the sample taken from the patient compared to the control level of said at least one marker, the at least one marker including C3, PLCB1, HLA-DRA, CD177, CCL5, PLCB2, CR1, NFKB, NCAM1/CD56, IFNG, IL-22, SELP, CASP1, PADI4, ITGB2, TLR2, CD14, CCL8, PDGFA, PDGFRAB, HIFα, IGFBP6, IGFBPL1, APP, JAM2, KCNH2, S100A14, KIAA0319, SNAPIN, and ITGAM.

In one embodiment of the method of diagnosing Long-COVID in a patient of the present disclosure, the at least one marker includes C3, PLC1, HLA-DR, CD177 and CCL5.

In another embodiment of the method of diagnosing Long-COVID in a patient of the present disclosure, the comparison is made using machine learning.

In another embodiment of the method of diagnosing Long-COVID in a patient of the present disclosure, when the subject is diagnosed has having Long-COVID, then the method further comprises treating the subject for Long-COVID.

In another embodiment, the present disclosure provides for a method of diagnosing a patient with Long-COVID, the method comprising comparing the level of resting natural killer cells in a sample taken from the patient to a control level resting natural killer cells in a sample of any one of a healthy control subject, a mild COVID-19 control subject and a severe COVID-19 control subject.

In another embodiment, the present disclosure relates to a method of treating a Long-COVID patient, the method comprising (a) providing a Long-COVID patient having an increased level of expression of at least one marker associated with a neurologic disorder relative to a healthy subject, and (b) treating the Long-COVID patient with a drug or agent effective to reduce the level of expression of the at least one marker associated with the neurologic disorder.

In one embodiment of the method of treating a Long-COVID patient of the present disclosure, the at least one marker associated with the neurologic disorder is at least one of S100A14, KIAA0319, SNAPIN, APP, JAM2, KCNH2, and IROR1.

In another embodiment of the method of treating a Long-COVID patient of the present disclosure, the marker is APP the neurological disorder is Alzheimer's disease, and the drug includes at least one drug or agent effective to prevent or treat Alzheimer's disease.

In another embodiment of the method of treating a Long-COVID patient of the present disclosure, the at least one drug or agent effective to prevent or treat Alzheimer's disease include Aducanumab, Gantenerumab, BAN2401, ALZ-801, Lecanemab, Bepranemab, Galantamine, rivastigmine and donepezil.

In another embodiment of the method of treating a Long-COVID patient of the present disclosure, the at least one marker is KCNH2, and the drug or agent is a KCNH2 inhibitor.

In another embodiment, the present disclosure relates to a method of treating a Long-COVID patient, the method comprising (a) providing a Long-COVID patient having a different level of expression of at least one marker associated with a cardiometabolic injury relative to the levels of said at least one marker in a healthy subject, and (b) treating the Long-COVID patient with a drug or agent effective to normalize the level of expression of the at least one marker.

In one embodiment of the method of treating a Long-COVID patient, the at least one marker includes FN1, CCL5, MMP7, MNDA and GPNMB.

In another embodiment of the method of treating a Long-COVID patient, the at least one marker is CCL5, and the drug or agent is a CCL5 inhibitor.

In another embodiment, the present disclosure provides for a method of treating a Long-COVID patient, the method comprising (a) providing a patient having at least one marker in the HIF signaling pathway upregulated, and (b) administering the patient with a drug effective to mediate the HIF signaling pathway such as to reduce the upregulation of the at least one marker.

In one embodiment of the method of treating a Long-COVID patient of the present disclosure, the at least one drug is selected from Daprodustat, Molidustat, Cindunistat, Icrucumab, Roxadustat, Firtecan pegol, Nesvacumab, Pabinafusp alfa, Vandetanib and Ponatinib.

In another embodiment of the method of treating a Long-COVID patient of the present disclosure, the markers known to be upregulated in Long-COVID include: IL-6, IFNγ, GF, IFNγR, RTK NFkB, elf4E, MEK, PI3K, AKT, PHD, HIF1β, TIMP1, CD18, EPO, TF, TFRC, VGEF, FLT1 (VEGFR1), EGF, ANGPT, EDN1, HK, ENO1, Bcl2, P21/p27.

In another embodiment, the present disclosure relates to a use of a drug effective to mediate the HIF signaling pathway in the treatment of a Long-COVID patient. In one embodiment, the drug is selected from Daprodustat, Molidustat, Cindunistat, Icrucumab, Roxadustat, Firtecan pegol, Nesvacumab, Pabinafusp alfa, Vandetanib and Ponatinib.

### BRIEF DESCRIPTION OF THE DRAWINGS

The following figures illustrate various aspects and preferred and alternative embodiments of the disclosure.

FIGS. 1A and 1B. Study model and dimensionality reduction of data sets. (1A) Model in the left panel illustrates four study cohorts, including Long-COVID, severe COVID-19, mild COVID-19, and healthy control subjects. The plasma proteome obtained by PEA, with the expression of ~3,000 plasma proteins measured. Right panel informs on the type of data processing strategy. (1B) Principal component analysis and hierarchical clustering. Unit variance scaling was applied to rows; SVD with imputation is used to calculate principal components. X and Y axis show principal component 1 and principal component 2 which explain 23.9% and 12.7% of the total variance, respectively (N=4 data points, the means of the values given by all patients within a group). Data was processed by Clustvis software (https://github.com/taunometsalu/ClustVis).

FIGS. 2A-2D. Natural killer (NK) cells change phenotypes in Long-COVID from activated to resting. (2A) General immune cell typing using CIBERSORT analysis tool. The algorithms were applied to plasma proteome data sets. Graphs show the proportion of each cell type compared to the healthy control cohort (HCTR). (2B) Diagram shows the NK cell cytotoxic pathways, a segment of KEGG pathways. Up-regulated biomarkers are shown with * and down-regulated (SLP76) with **. Arrows show direct interactions. Analysis was done with KEGG Mapper, also confirmed by iPathwayGuide. (2C) Heatmaps represent the profiling of the NK cell phenotype. Left heatmap shows the hierarchical clustering of all hand curated markers and in the right panel the similarity test based on Pearson Correlation. For data visualization we used Morpheus software from Broad Institute. Graphs show individual NK cell markers expression in plasma compared among study groups. Statistical significance was processed with GraphPad 9, P-value was considered significant if <0.05 in ANOVA (group). (2D) Left graph represents pathways analysis scoring done with iPathwaysGuide; tumor necrosis factor (TNF) signaling was the highest hit; schematic below represents the main predicted interactions of ANGPT1 (STRING analysis confirmed by iPathways guide). Graph in the middle shows the TNF expression in plasma as detected by PEA. The rest of the graphs indicate the plasma expression levels of Angiopoietin 1 (ANGPT1), Matrix metalloproteinase 9 (MMP9) and Vasculo-Endothelial Growth Factor A (VEGFA); all these proteins are predicted to be induced and potentiated by TNF. Statistical significance was processed with GraphPad 9, P-value was considered significant if <0.05 with Mann-Whitney U test.

FIGS. 3A-3D. Neutrophil extracellular trap formation has a critical role in Long-COVID. (3A) Diagram shows the NK cell cytotoxic pathways mapped on KEGG charts. (3B) All boxes represent up-regulated biomarkers (KEGG Mapper, also confirmed by iPathwayGuide software). Graphs display the levels of individual NET marker expression in plasma among the study cohorts. Statistical significance was determined with GraphPad 9, P-value was considered significant if <0.05 with an ANOVA. (3C) Heatmaps represent the profiling of the neutrophil cell phenotype. Markers have been manually curated. In the left panel, heatmap shows the hierarchical clustering of all markers and in the right panel the similarity tmatrix based on Pearson Correlation. For data visualization we used Morpheus software from Broad Institute was used. (3D) Graphs show individual neutrophil cell markers expression in plasma; comparison among all study cohorts. Statistical significance was processed with GraphPad 9, P-value was considered significant if <0.05 with ANOVA.

FIGS. 4A-4B. Silent TGFβ1 signaling and reduced EP300 levels may repurpose TNF actions in Long-COVID. (4A) Diagram shows TGFβ signaling pathways. *: Up-regulated biomarkers and **: down-regulated (KEGG Mapper, confirmed by iPathwayGuide software). (4B-4C) Graphs represent the expression levels of markers associated with the TGFβ1 pathway. Statistical significance was established using GraphPad-9, and P-value was considered significant if <0.05 with Mann-Whitney U test. Key protein interactions are presented in the diagram from the middle panel (right side), an output of STRING confirmed with iPathwayGuide software. The down regulation of the TGFβ1 seem to favor TNF action onto EP/p300 and perhaps NFκB.

FIGS. 5A-5B. COVID-19 associated hypoxia is mediated by HIF1 and EP300, potentially disrupting the vascular bed. Long-COVID data has been intersected with Mild and Severe COVID-19 data sets, each normalized to the healthy control group (meta-analysis was performed with KEGG Mapper and confirmed by iPathwaysGuide software). Venn digram at the left shows the intersection of the total number of signaling pathways among the clinical groups (iPathwayGuide). (5A) One of the pathways predicted to support vasculo-proliferative disease was HIF1-signaling pathway. Diagrams represent sequences of the HIF1 pathways mapped onto KEGG charts and analyzed by iPathwayGuide software. The configuration of this pathway evolved from Mild COVID-19 to Severe COVID-19, having the most prominent expression in Long-COVID. Note: vasculo-proliferative disease is mediated by HIF, which increases both proliferation and angiogenesis. (5B) Table shows the drugs associated with the HIF pathway which can be repurposed for Long-COVID therapeutics (drug Bank output).

FIGS. 6A-6C. Long-COVID is associated with cancer-related proliferation pathways, largely associated with the vascular bedding status. (6A) Diagram at the left shows a cancer related signaling pathway mapped on KEGG charts (KEGG Mapper). The top right panel presents graphs depicting the expression of individual markers that belong to the proliferative pathways mediated by VEGF. Comparison has been done among all study groups and statistical significance was established using GraphPad-9, with P-value considered significant if <0.05 with ANOVA. (6B) Heatmaps reflect global expression levels of the growth-factors that could be responsible for the cell proliferation via receptor tyrosine kinases (RTKs) membrane receptors. Markers have been manually curated based on literature reports. In the left panel heatmap shows the hierarchical clustering of all markers and in the right panel the similarity matrix based on Pearson Correlation. For data visualization we used Morpheus software from Broad Institute. (6C) Graphs measure the levels of the insulin-like growth factor system (IGF), molecules known to regulate angiogenesis but also to ensure the sustainability of the vascular network. Statistical significance was established using GraphPad-9, and P-value was considered significant if <0.05 with ANOVA.

FIGS. 7A-7C. Long-COVID impacts brain functionality through vasculo-proliferative events hosted by the blood-brain barrier. (7A) Heatmaps reflect the levels of neurological markers per OLINK panels I, II and III, which were largely changed in Long-COVID patients. Markers have been curated by OLINK. Values have been hierarchically clustered based on Pearson correlation algorithms. For data visualization we used Morpheus software, a tool designed by the Broad Institute. (7B) Functional annotation was performed with tools from the Gene Set Enrichment Analysis (GSEA) platform. These clusters refer to leucocyte migration, positive immune signals, glial cell differentiation, neurogenesis and MAPK regulatory pathways. At the right, diagram shows a segment of brain degeneration pathways as described by KEGG charts and processed for protein-protein interaction capacities with STRING software (confirmed by iPathwayGuide). APP and TNF biomarkers are highlighted as major players. All together these data point to a brain-blood barrier dysfunctionality based on cell proliferation. (7C) The expression levels of markers from the above functional groups have been plotted on graphs, where comparison has been done among all study groups and statistical significance was determined by GraphPad-9. P-value was considered significant if <0.05 with ANOVA. One highly expressed biomarker marker was the Amyloid Precursor Protein (APP/Presenilin) that is known as a pathognomonic marker for Alzheimer disease and brain inflammation.

FIGS. 8A-8D. Long-COVID is associated with cardiometabolic damage associated with vasculo proliferative events. (8A) Heatmaps reflect the levels of cardio-metabolic markers (per OLINK panels I and II) that were changed in Long-COVID patients. Markers have been curated by OLINK. Values have been hierarchically clustered based on Person correlation algorithms. For data visualization we used Morpheus software, a tool designed by the Broad Institute. (8B) Post-hierarchical analysis these markers formed three functional clusters (#1, 2 and 3) determined with tools from the GSEA platform. These clusters refer to extracellular matrix remodeling, cell adhesion and motility, and angiogenesis (possible tube formation), respectively. (8C) Markers forming the three functional clusters were analyzed for protein-protein interaction using STRING software (Search Tool for the Retrieval of Interacting Genes/Proteins). This analysis shows that extracellular matrix remodeling is dominated by integrin interactions and calcium binding events, as well as dysregulated macrophage activity (also observed by CIBERSORT analysis). (8D) The expression levels of the markers that interact directly was plotted on graphs, where comparison has been done among all patient cohorts and statistical significance was determined by GraphPad-9 (P-value was considered significant if <0.05 with ANOVA).

FIG. 9. Schematic summary of the Long-COVID-19 pathology as reflected in plasma proteome.

### DESCRIPTION

### Abbreviations

Unless defined otherwise, all technical and scientific terms used herein have the same meaning as commonly understood by one of ordinary skill in the art to which this disclosure belongs. Also, unless indicated otherwise, except within the claims, the use of “or” includes “and” and vice versa. Non-limiting terms are not to be construed as limiting unless expressly stated or the context clearly indicates otherwise (for example “including”, “having” and “comprising” typically indicate “including without limitation”). Singular forms including in the claims such as “a”, “an” and “the” include the plural reference unless expressly stated otherwise. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this disclosure.

The contents of all documents (including patent documents and non-patent literature) cited in this application are incorporated herein by reference.

All numerical designations, e.g., levels, amounts and concentrations, including ranges, are approximations that typically may be varied (+) or (−) by increments of 0.1, 1.0, or 10.0, as appropriate. All numerical designations may be understood as preceded by the term “about”.

“COVID-19 subjects” or “COVID-19 patients” are subjects who are acutely ill with confirmed SARS-COV-2 positive. In the Examples, the studies have been carried out with ward COVID-19 subjects (mild/moderate disease) and with intensive care unit COVID-19 subjects (severe disease or critically ill).

The term “level” covers either one or both levels of expression of a marker and/or levels of concentration of a marker, or the number or percentage of cells.

The term “subject” as used herein refers all members of the animal kingdom including mammals, preferably humans.

The term “patient” as used herein refers to a subject that is COVID-19 or Long-COVID.

“Plasma” is the clear, straw-colored liquid portion of blood that remains after red blood cells, white blood cells, platelets and other cellular components are removed.

The term “pharmaceutically acceptable carrier”, “pharmaceutically acceptable excipient”, “physiologically acceptable carrier”, or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

Vascular proliferation, or angiogenesis, is a multistep process for the formation of new blood vessels. “Vaso-proliferative proteins” or “angiogenic proteins” refer to proteins that lead to activation of the cellular pathways that result in angiogenesis. Proteins, including angiogenic proteins, can be measured with antibody tests (i.e., Western blotting, Luminex bead-based assays, Proximity Extension Assay (PEA), planar multiplex assays, lateral flow assays, electrical conductivity devices, electrochemiluminescence, proximal extension assay with oligonucleotide-labeled antibodies, ELISA and RIA), flow cytometry or mass spec techniques. Enzymes can be measured with enzyme assays that measure either the consumption of a substrate or production of product over time. Differential expression profiles may have important diagnostic value, even in the absence of specifically identified proteins. Single protein spots can then be detected, for example, by immunoblotting, multiple spots or proteins using protein microarrays. The term “proteomic profile” is used to refer to a representation of the expression pattern of a plurality of proteins in a biological sample, e.g., a biological fluid at a given time. The proteomic profile can, for example, be represented as a mass spectrum, but other representations based on any physicochemical or biochemical properties of the proteins are also included. Thus, the proteomic profile may, for example, be based on differences in the electrophoretic properties of proteins, as determined by two-dimensional gel electrophoresis, e.g., by 2-D PAGE, and can be represented, e.g., as a plurality of spots in a two-dimensional electrophoresis gel. The proteomic profile typically represents or contains information that could range from a few peaks to a complex profile representing 50, 1,000 or more peaks. Thus, for example, the proteomic profile may contain or represent at least 2, or at least 5 or at least 10 or at least 15, or at least 20, or at least 25, or at least 30, or at least 35, or at least 40, or at least 45, or at least 50 proteins, or over 1,000 proteins.

Any suitable point-of-care measurement devices can be used to measure the proteins of the present disclosure, including testing with portable, table/counter-top, hand-held, lateral flow device (including lateral flow immunochromatographic assay including those described in WO/2022/236120, the content of which is incorporated herein by reference), chip or MS protein testing instruments.

The terms “agent”, “drug”, “therapeutic agent”, and “chemotherapeutic agent” refer to a compound, or a pharmaceutical composition thereof, or an inhibitor, or an antibody which is administered to a subject for treating, preventing, or ameliorating one or more symptoms of Long-COVID pathology and disorders that are linked to or associated with or that result from Long-COVID.

“Long-COVID” refers to subjects not recovering for several weeks or months following the start of symptoms that were suggestive of COVID-19 and to survivors of COVID-19 that suffer diffuse symptoms that can persist for at least two months. According to the CDC website “Many post-COVID conditions can be improved through already established symptom management approaches (e.g., breathing exercises to improve symptoms of dyspnea). Creating a comprehensive rehabilitation plan may be helpful for some patients and might include physical and occupational therapy, speech and language therapy, vocational therapy, as well as neurologic rehabilitation for cognitive symptoms. A conservative physical rehabilitation plan might be indicated for some patients (e.g., persons with post-exertional malaise); consultation with physiatry for cautious initiation of exercise and recommendations about pacing may be useful. Gradual return to exercise as tolerated could be helpful for most patients. Optimizing management of underlying medical conditions might include counseling on lifestyle components such as nutrition, sleep, and stress reduction (e.g., meditation).” [Taken from CDC website: www.cdc.gov]

The term “marker” or “biomarker” refers to an indicator, including biological molecules (i.e., proteins, lipids, nucleic acids, sugars, and so forth) which could provide organ-specific diagnosis or prognosis, including organ specific diagnosis or prognosis, in Long-COVID patients.

A “prognosis” is the likely course and outcome of a disease. The prognosis may include the likelihood of or risk for a Long-COVID patient of developing a neurologic dysfunction or a cardiac event.

Samples include blood, blood plasma, blood serum, capillary blood/plasma, venous blood, saliva, synovial fluid, urine, spinal fluid, bronchoalveolar lavage, sweat, tears, breath samples and extracts.

### Overview

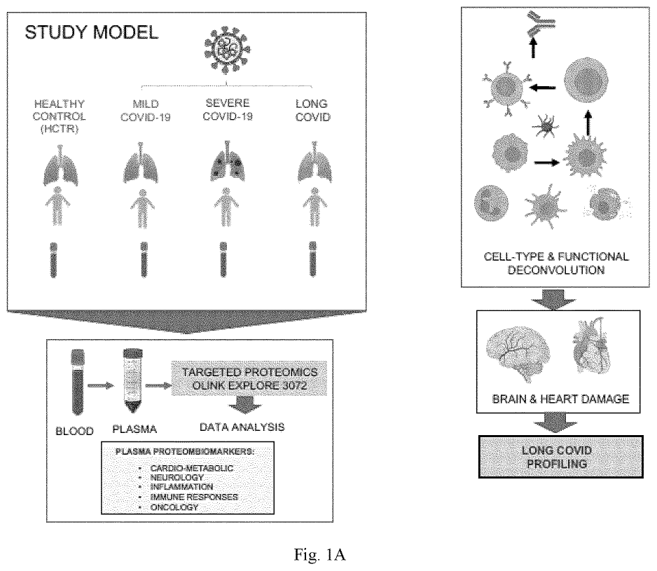

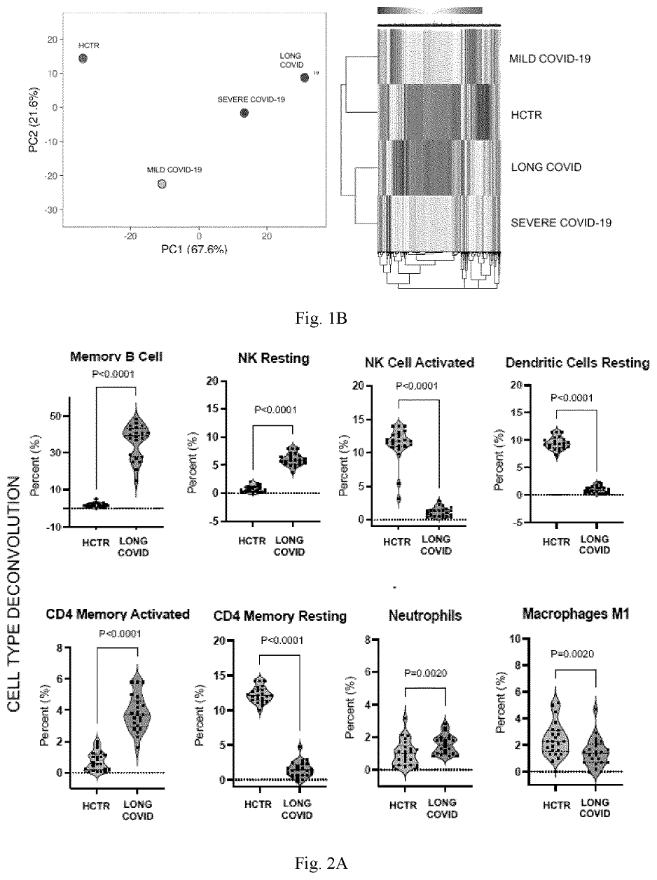

As illustrated in FIG. 1B Long-COVID is a distinct disease compared to COVID-19 ICU patients (severe disease), although the cohorts did share some mechanisms. In contrast, the COVID-19 medical ward inpatients (mild disease) shared mechanisms closer to the healthy control subjects.

In embodiments the present disclosure relates to biomarkers of Long-COVID and therapeutic targets for the prevention and/or treatment of Long-COVID.

In embodiments the present disclosure relates to organ-specific prognosis of Long-COVID patients/subjects. In other embodiments this disclosure relates to methods of treating Long-COVID patients. It has been unexpectedly found that Long-COVID patients express markers for brain dysfunctions and cardiovascular dysfunction.

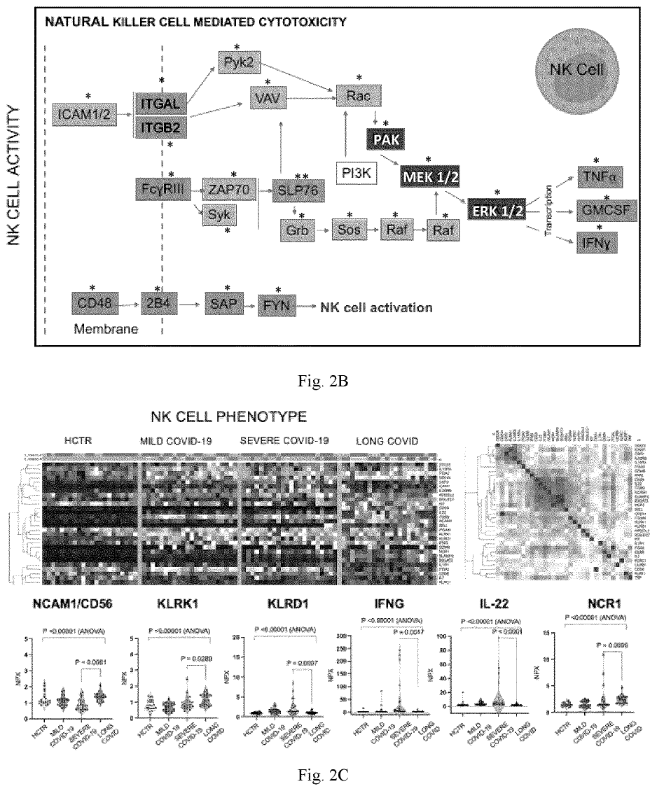

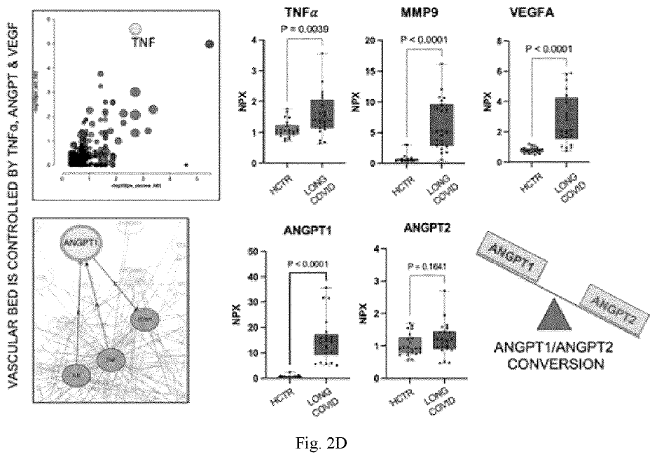

Natural killer (NK) cells from Long-COVID disease changed phenotype from activated to resting (FIG. 2A).

In one embodiment, the present disclosure provides for biomarkers of Long-COVID that serve to diagnose Long-COVID.

In one embodiment, the markers of Long-COVID are listed in Table 1. Levels of expression of the markers listed in Table 1 are statistically different from the levels of expression of each marker in HCTR, mild COVID-19 and Severe COVID-19. Markers labeled with “*” are markers that are not significantly different between healthy control, mild COVID-19 and severe COVID-19. Markers labeled with “*” serve to uniquely distinguish Long-COVID.

TABLE 1

| | Expression in |
|---|---|
| Marker | Long-COVID¹ |
| C3* | Increased |
| PLCB1* | Increased |
| HLA-DR* | Increased |
| CD177* | Increased |
| CCL5* | Increased |
| NFKB | Increased |
| NCAM1/CD56 | Increased |
| IFNG | Increased |
| IL-22 | Increased |
| PLCB2 | Increased |
| SELP | Increased |
| CASP-1 | Increased |
| PADI4 | Increased |
| CR1 | Increased |
| ITGB2 | Increased |
| TLR2 | Increased |
| CD14 | Increased |
| CCL8 | Increased |
| PDGFA | Increased |
| PDGFRAB | Increased |
| HIFα | Increased |
| IGFBP2 | Decreased |
| IGFBP6 | Increased |
| IGFBPL 1 | Increased |
| APP | Increased |
| JAM2 | Increased |
| KCNH2 | Increased |
| S100A14 | Increased |
| KIAA0319 | Increased |
| SNAPIN | Increased |
| FN1 | Decreased |

¹relative to healthy control, mild COVID-19 and severe COVID-19

As such, in one embodiment the present disclosure provides for a method of diagnosing a patient with Long-COVID, the method comprising comparing the level, such as the level of expression, of at least one of the markers listed in Table 1 measured in a sample taken from the patient to a control level, such as a level of expression, of said at least one marker in a sample taken of any one of a healthy control subject, a mild COVID-19 control subject and a severe COVID-19 control subject. In one embodiment, the at least one marker include C3, PLCB1, HLA-DR, CD177 and CCL5.

### Machine Learning

In one embodiment, machine learning techniques are used for diagnosing Long-COVID using levels of expression of at least one biomarker in a biological sample. In machine learning, preconceived expectations are averted and through a process of optimization, a model emerges, is fitted, and fine-tuned, connecting inputs-levels of expression in a biological sample of at least one biomarker in this case- and outputs-Long-COVID versus control, mild COVID-19 and/or severe COVID-19 categorization here.

Examples of suitable machine learning techniques or models include, for example, Supervised Learning, Unsupervised Learning, and Reinforcement Learning. Examples of machine learning algorithms include linear regression, decision trees, random forest (RF) classifiers, and XGBoost.

The machine learning technique is trained with a training data set such as the levels of expression of at least one biomarker of known Long-COVID positive controls and/or with levels of expression of at least one biomarker of Long-COVID negative controls (one or more of healthy controls, mild COVID-19 and/or severe COVID-19). The Long-COVID positive controls and the Long-COVID negative controls are obtained, in one embodiment, from libraries of expression of at least one marker in Long-COVID positive patients and in Long-COVID negative patients. A diagnosis of the subject/patient is obtained by testing the trained machine learning technique with the levels of expression of the at least one biomarker in a biological sample taken from the subject. In one embodiment, the positive and negative Long-COVID controls may be obtained for example through clinical information and standard clinical measurements by medical specialists.

In embodiments, the training data set further includes control data of different stages COVID-19 (i.e., mild COVID-19 and severe COVID-19) thus allowing classifying the COVID-19 stage of the subject/patient.

### Therapy

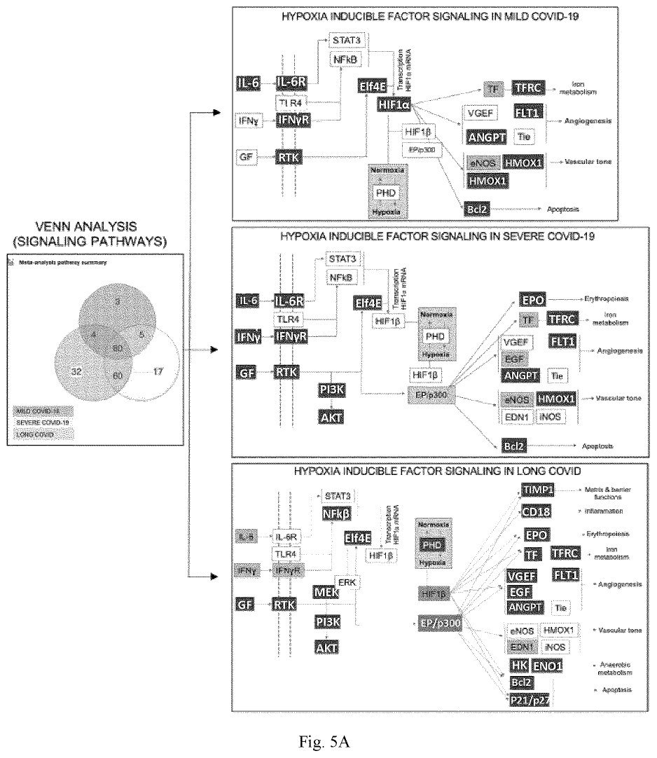

In another embodiment, with reference to FIG. 5A, the Applicant demonstrates that Long-COVID shares 80 pathways with mild COVID-19 (non-severe COVID-19) and severe COVID-19, shares 60 pathways with severe COVID-19 and shares 4 pathways with mild COVID-19. Long-COVID retains 32 unique or independent pathways. The HIF signaling pathway (FIG. 5A) has been shown by the Applicant to progress proportionally with disease severity, to a maximum in Long-COVID (see FIG. 5A). FIG. 5B lists drugs that can regulate the HIF pathway.

In one embodiment, the present disclosure relates to the use of at least one of the drugs listed in FIG. 5B to regulate the HIF pathway in the treatment of Long-COVID.

In one embodiment, the present disclosure provides for a method of treating Long-COVID in a patient, the method comprising (a) measuring the level of expression of at least one marker in the HIF signaling pathway known to be upregulated in Long-COVID in the patient, and when the level of expression of the at least one marker is overexpressed relative to healthy subjects (b) treat the patient with at least one drug that mediates the HIF signaling pathway such as to reduce the level of expression of the at least one marker.

In one embodiment, the drug/agent is selected from the drugs listed in FIG. 5B, i.e., Daprodustat, Molidustat, Cindunistat, Icrucumab, Roxadustat, Firtecan pegol, Nesvacumab, Pabinafusp alfa, Vandetanib and Ponatinib.

The markers known to be upregulated in Long-COVID include: IL-6, IFNγ, GF, IFNγR, RTK NFkB, elf4E, MEK, PI3K, AKT, PHD, HIF1β, TIMP1, CD18, EPO, TF, TFRC, VGEF, FLT1 (VEGFR1), EGF, ANGPT, EDN1, HK, ENO1, Bcl2, P21/p27.

### Neurologic Dysfunction

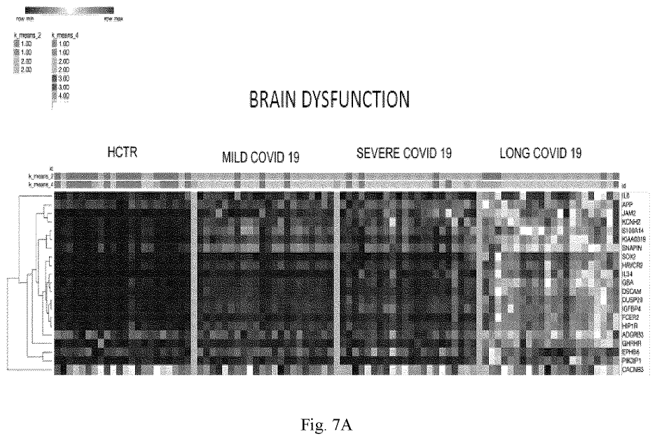

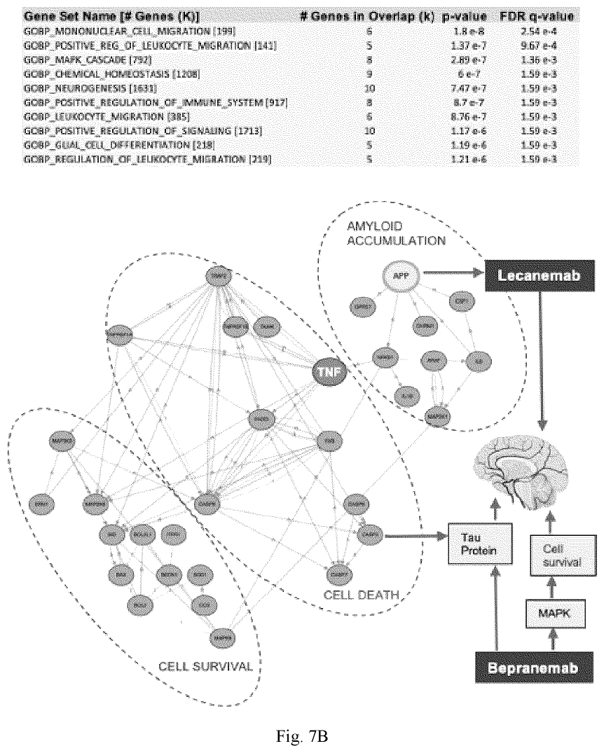

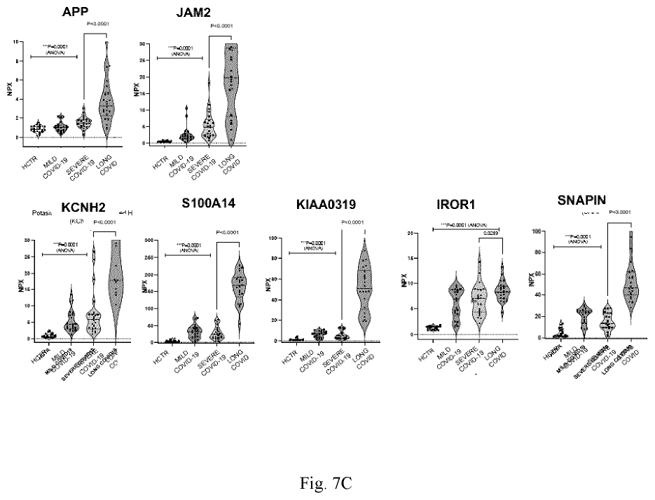

It has been unexpectedly found that Long-COVID patients are at an elevated risk for developing a neurological disorder. As shown in FIG. 7A, heatmaps reflect the levels of neurological markers per OLINK panels I, II and III, which were largely changed in Long-COVID patients relative to healthy control subjects, mild COVID-19 patients, and severe COVID-19 patients. The markers listed in FIG. 7A are listed in Table 2. As shown in FIG. 7C, when compared to healthy subjects, mild COVID-19 patients and severe COVID-19 patients, Long-COVID patients show an increased level of KIAA0319, S100A14, APP, JAM2, KCNH2, IROR1 and SNAPIN.

TABLE 2
Markers of FIG. 7A

| | | |
|---|---|---|
| IL6 | SOX2 | FCER2 |
| APP | HAVCR2 | HP1R |
| JAM2 | IL34 | ADGRB3 |
| KCNH2 | GBA | GHRHR |
| S100A14 | DSCAM | EPH86 |
| KIAA0319 | DUSP29 | PIK3IPI |
| SAPIN | IGFBP4 | CACNB3 |

In embodiments, the present disclosure provides for a method of determining a risk of developing a neurological disorder in a Long-COVID patient comprising (a) testing levels, such as the levels of expression, of at least one marker associated with a neurologic disorder in a Long-COVID patient, and (b) making a determination that the Long-COVID patient is at risk of developing said neurological disorder when the level of expression of said at least one marker is increased in the Long-COVID patient compared to the levels of expression of said at least one marker in a reference healthy control subject. In one embodiment, the at least one marker includes at least one of the markers listed in Table 2. In another embodiment, the at least one marker is at least one of KIAA0319, S100A14 and SNAPIN. In embodiment, the determination is made using machine learning techniques.

In another embodiment, the present disclosure provides for a method of treating a Long-COVID patient for a neurologic disorder, the method comprising (a) providing a Long-COVID patient having an increased level of expression of a marker associated with the neurologic disorder relative to a healthy subject, and (b) treating the Long-COVID patient with a drug or agent effective against the neurologic disorder.

In embodiments, the marker associated with the neurologic disorder is at least one of the markers listed in Table 2.

In embodiments, the marker associated with the neurologic disorder is at least one of APP, JAM2, KCNH2, S100A14, KIAA0319, IROR1 and SNAPIN.

One of the highly expressed markers in Long-COVID patients was the amyloid precursor protein (APP) which is known to be a pathognomonic marker for both Alzheimer disease and brain inflammation [62-66] (see FIGS. 7A and 7C). Additional markers for brain dysfunction include JAM2 (endothelial tight junctions protein), SNAPIN (a mediator of neuronal autophagy-lysosomal function in developing neurons), KCNH2 (potassium voltage-gated ion channel), S100A14 (involved in cell motility adhesion and growth), KIAA0319 (language impairment biomarker), and IROR1 (a receptor tyrosine kinase like orphan receptor 1, which regulates neurites growth in the central nervous system having also WNT-signaling pathway functions, and being crucial for the auditive apparatus maintenance).

In one embodiment, the Long-COVID patient has an increased expression of APP, placing the Long-COVID patient at an increased risk of developing Alzheimer's disease (AD). In this embodiment, the Long-COVID patient is treated with a drug effective for preventing or treating AD or with a drug that helps manage symptoms of AD. Example of drugs effective for preventing or treating AD include Aducanumab, Gantenerumab, BAN2401, ALZ-801, Lecanemab and/or Bepranemab for Mild Cognitive Impairment or Early-Stage AD. Drugs that help manage symptoms of AD include Galantamine, rivastigmine and donepezil.

Mutations and variants of the KCNH2 gene are one cause of congenital long QT syndrome (LQTS). Examples of KCNH2 inhibitors include amiodarone, astemizole and its metabolite desmethylastemizole, cisapride, disopyramide, dofetilide, erythromycin, fluoxetine, grepafloxacin, haloperidol half maximal inhibitory concentration (IC50) approximately 63 nmol/l, hydroxyzine, ibutilide, levomethadyl, lidoflazine IC50 less than 37 nmol/l, methadone; mibefradil, moxifloxacin, perhexiline, pimozide IC50 approximately 18 nmol/l, prenylamine IC50 approximately 590 nmol/l, probucol, quinidine; risperidone IC50 167 nmol/l, sertindole IC50 approximately 3 nmol/l, IC50 approximately 210 nmol/l, sotalol, telithromycin, terfenadine IC50 less than 52 nmol/l, terodiline, thioridazine IC50 approximately 191 nmol/l, IC50 approximately 224 nmol/l, ziprasidone IC50 approximately 169 nmol/l. Weak inhibitors of KCNH2 (IC50>1 μmol/l) include arsenic trioxide IKr approximately 300 μmol/l, chlorpheniramine IC50 approximately 13 μmol/l, cimetidine IC50 greater than 10 μmol/l, doxepin IC50 approximately 4 μmol/l, loratadine IC50 approximately 4 μmol/l, lovastatin IC50 approximately 7 μmol/l, olanzapine IC50 approximately 6013 nmol/l, pentamidine Iherg approximately 1 mmol/l, procainamide IC50 approximately 139 μmol/l, pyrilamine IC50 approximately 6 μmol/l, quetiapine IC50 approximately 5765 nmol/l, sparfloxacin (fluoroquinolone) IC50 approximately 18 μmol/l. Drugs that prolong QT interval by reducing cell surface KCNH2 expression include pentamidine, arsenic trioxide [Taken from Connie Oshiroa, et al., Pharmacogenet Genomics. 2010 December; 20 (12): 775-777. Doi: 10.1097/FPC.0b013e3283349e9c).

### Cardiometabolic Injury

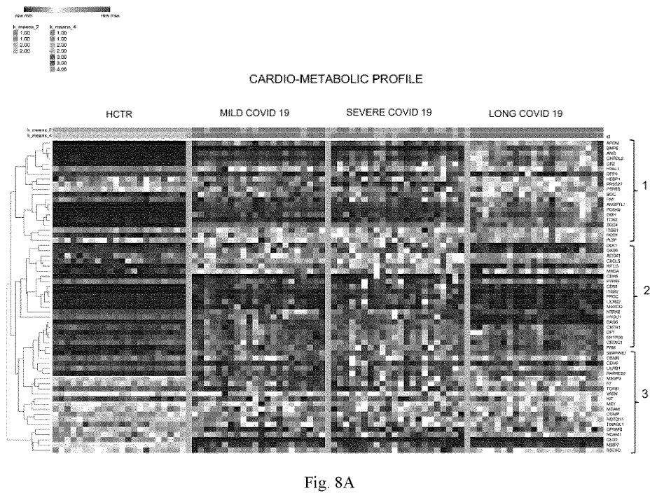

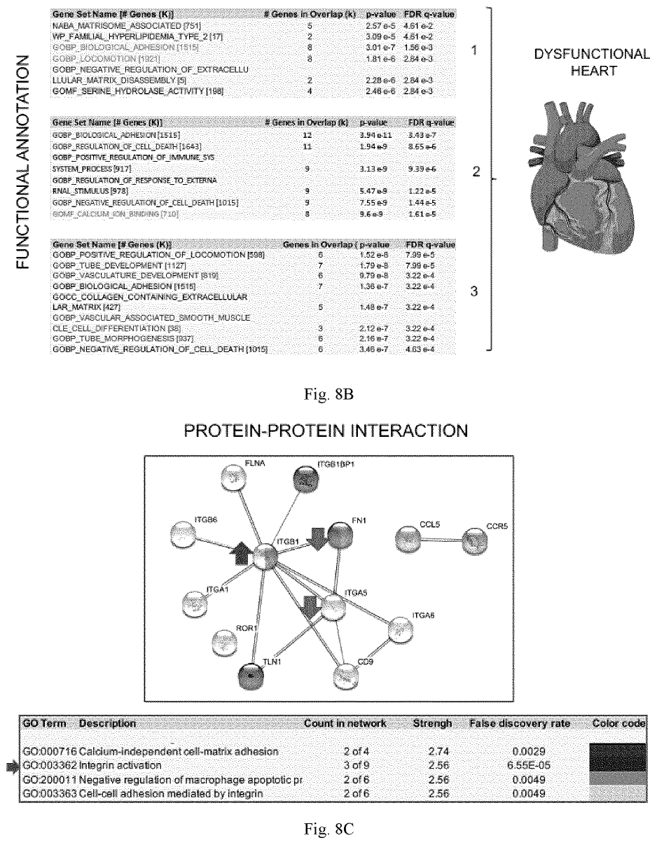

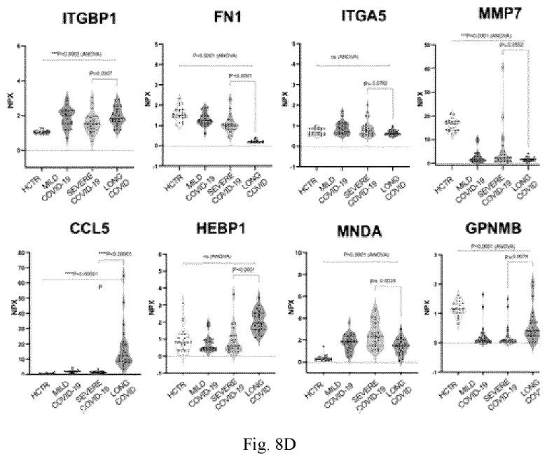

It has been unexpectedly found that Long-COVID patients are at an elevated risk for developing cardiometabolic injury. Heatmaps reflect the levels of cardio-metabolic markers (per OLINK panels I and II) that were changed in Long-COVID patients. These markers are listed in Table 3. As shown in FIG. 8D, when compared to healthy subjects, Long-COVID patients show statistically different levels of ITGBP1 (increased in Long-COVID), FN1 (decreased in Long-COVID), MMP7 (decreased in Long-COVID), CCL5 (increased in Long-COVID), MNDA (increased in Long-COVID) and GPNMB (decreased in Long-COVID).

TABLE 3
Markers listed in FIG. 8A

| | | | | | | |
|---|---|---|---|---|---|---|
| APOM | BOC | DLK1 | PROC | PAM | KIT | SSC5D |
| BMP6 | FAP | GAS6 | LILRB2 | SERPINE1 | MET | |
| ANG | ANGPTL1 | ACOX1 | MARCO | OSMR | MCAM | |
| CHRDL2 | PCSK9 | CXCL5 | NTRK2 | CDH6 | COMP | |
| CR2 | GGH | KITLG | HYOU1 | LILRB1 | NOTCH1 | |
| HYAL1 | TCN2 | MNDA | BAG6 | RARRES2 | TINAGL1 | |
| DPP4 | SOC4 | CDH5 | CNTN1 | MEGF9 | GPNMB | |
| HEBP1 | ITGB1 | PIPRF | DPT | F7 | NCAM1 | |
| PRSS27 | ROR1 | CD93 | ENTPD6 | TGFB1 | GLO1 | |
| PTPAS | PLTP | ITGB2 | CRTAC1 | VASN | MMP7 | |

In one embodiment, the present disclosure provides for a method of determining a risk of developing a cardiometabolic injury in a Long-COVID patient comprising (a) testing/measuring the levels of at least one marker associated with a cardiometabolic injury in a Long-COVID patient, and (b) making a determination that the Long-COVID patient is at risk of developing said cardiometabolic injury when the levels of said marker in the Long-COVID patient is different to the levels of said marker in a healthy subject.

In one embodiment of the method of determining a risk of developing a cardiometabolic injury in a Long-COVID patient of the present disclosure, the at least one marker associated with the cardiometabolic injury are FN1 and CCL5, and the determination that the Long-COVID patient is at risk of developing said cardiometabolic injury is made when the level (such as the level of expression) of FN1 goes down and the level (such as the level of expression) of CCL5 goes up in the Long-COVID patient relative to the level of FN1 and the level of CCL5 in the reference healthy control subject.

In another embodiment, the present disclosure provides for a method of treating a Long-COVID patient for a cardiometabolic injury, the method comprising (a) providing a Long-COVID patient having a different level of expression of a marker associated with the cardiometabolic injury relative to a healthy subject, and (b) treating the Long-COVID patient with a drug or agent effective against the cardiometabolic injury.

In embodiments, the marker associated with the cardiometabolic injury is at least one of the markers of Table 3.

In embodiments, the marker associated with the cardiometabolic injury is at least one of FN1, CCL5, MMP7, HEBP1, MNDA and GPNMB (see FIG. 8D).

### Therapeutic Angiogenesis

1. Therapeutic Angiogenic Drugs (Li V W, Kung E F, Li W W. Molecular Therapy for Wounds: Modalities for stimulating Angiogenesis and Granulation. Manual of Wound Management (Bok Lec, Editor) McGraw Hill, 2004, p. 17-43; Li W, Talcott K, Zhai A, Kruger E, Li V. The Role of Therapeutic Angiogenesis in Tissue Repair and Regeneration Adv Skin Wound Care 2005; 18:491-500; Smiell J M, Wieman T J, Steed D L, et al. Efficacy and safety of becaplermin (recombinant human platelet-derived growth factor-BB) in patients with nonhealing, lower extremity diabetic ulcers: a combined analysis of four randomized studies. Wound Repair Regen. 1999; 7:335-346):
Growth factor-based therapies include the only FDA-approved recombinant protein drug recombinant human Platelet-derived growth factor (rhPDGF) (becaplermin, REGRANEX® 0.01% gel), which is indicated for diabetic neuropathic lower extremity ulcers.
Growth factors can also be delivered through autologous isolates of patient platelets such as Autologel, SmartPReP.
Currently, there are no FDA-approved angiogenic drugs for the treatment of ischemic cardiovascular disease.
Some early-stage clinical trials of therapeutic angiogenic agents have demonstrated reductions in symptoms of angina, increase in ability to exercise, and objective evidence of improved perfusion and left ventricular function following therapy.
Therapeutic Angiogenesis Promoting Devices: Negative pressure wound therapy (NPWT) such as the Vacuum Assisted Closure (V.A.C.) system induces angiogenesis through tissue microdeformations and mechanochemical coupling and signal transduction; MIST ultrasound is a low-frequency and low-intensity non-contact device that results in cell stimulation and increased wound perfusion; Hyperbaric Oxygen (HBO) promotes angiogenesis and wound healing by increasing Vascular endothelial growth factor (VEGF) expression and recruiting endothelial progenitor cells.
Cell-Based Therapies:
Tissue engineered products approved by the FDA include the bilayered skin substitute Grafstkin (Apligraf®) and the fibroblast dermal skin substitute Dermagraft. These products contain living or cryopreserved cells on a matrix capable of secreting and releasing multiple angiogenic growth factors into the wound bed.
CD34+ endothelial progenitor cells (EPC) derived from bone marrow or from peripheral blood have been found to enhance angiogenesis in ischemic tissues, increase transcutaneous oxygen, improve ankle-brachial index (ABI), increase collateral vessels by angiography and improve healing of leg ulcers.
Integra® Dermal Regeneration Template is an advanced skin replacement matrix that consists of a complex three-dimensional porous matrix that acts as a scaffold for cell migration and allows for regeneration of the dermal layer of the patient's skin. It can be used for Diabetic Foot Ulcers.

2. VEGF/VPF (Tan, Q., et al., European Journal of Cardio-thoracic Surgery 31 (2007) 806-811; vascular permeability factor (VPF).

3. ANG1/TIE1 pathway—ANG1 has vasculoprotective effects. It enhances the stability of newly formed vessels, inhibits vascular permeability induced by several inflammatory cytokines and attenuates pathological responses, including fibrosis. Recently, the angiopoietin (ANG)-TIE signaling pathway has emerged as an attractive vascular drug target. The ANG-TIE pathway is required for lymphatic and blood vessel development.
1. Recombinant/viral vectors ANG1 (Angiopoietin 1)
2. ANG2 (Angiopoietin 2) inhibitors (ANG2 blocks angiogenesis)

4. Statin therapy
Low-dose statin therapy may promote angiogenesis via multiple mechanisms, including enhanced NO production, augmented VEGF release, and activation of the Akt signaling pathway

5. Therapeutic/Prescribed Exercise
Exercise stimulates angiogenesis in skeletal muscle and heart. A lack of exercise leads to capillary regression.

6. Plasminogen Activator system (Plasmin)
https://www.news-medical.net/health/Angiogenesis-Stimulation.aspx
Plasmin also activates Matrix metalloproteinases (MMPs) such as MMP-1, MMP-3, and MMP-9. These are metalloproteinases.

6. Growth Factors (Fibroblast growth factor 2 (FGF2) and VEGF)
FGF 2 is vital for angiogenesis. It induces multiplication and movement of the cells as well as uPA production by endothelial cells. FGF-2 induces tube formation in collagen gels and alters integrin expression that helps in angiogenesis.

7. Combination therapies—VEGF, FGF, MMP1, plasma, etc. (Sabra, M., et al., Int. J. Mol. Sci. 2021, 22, 3722)

8. Stimulators of angiogenesis: VEGF, FGF, Hepatocyte Growth Factor (HGF), Angiopoietin 1 (Ang1) and Angiopoietin 2 (Ang2), Platelet-derived growth factors (PDGFs), insulin-like growth factor (IGF), Endoglin Interleukin 8, Thyroxin, VE-cadherin, Granulocyte colony-stimulating factor (G-CSF), Integrins, Ephrin, Endothelial nitric oxide synthase (eNOS), Transforming growth factor beta (TGFbeta), YKL40, HIF1α (Hypoxia Inducible Factor 1 Subunit Alpha), HDGF (Heparin Binding Growth Factor), Notch/DLL4 (Delta-like 4), Semaphrorins.

9. Chinese herbal medicines. Chinese herbal medicines that target angiogenesis can provide therapeutic effect, including active components Salvianolic acid A, Tanshinone IIA, Ferulaic acid, Rhodiola, Salidroside, Astragalosides, Berberine, Puerarin and Extract of Geum japonicum. These active components can be obtained from Radix Salvia miltiorrhiza, Radix Angelica Sinensis, Rhizoma Rhodiolae Kirilowii, Shanxi Astragalus membranaceus, Berberis and Berberis aristate, Radix Puerariae, Germ japonicum (Dongqing Guo, et al., Frontiers in Pharmacology, (2018) 9, 428).

10. CCL5 Inhibitors: C—C chemokine ligand 5 (CCL5), also known as RANTES, exerts proangiogenic effects by promoting endothelial cell migration, spreading, neovessel formation, and vascular endothelial growth factor (VEGF) secretion (Donatella Aldinucci and Naike Casagrande, Int. J. Mol. Sci. 2018, 19, 1477). Inhibitors of CCL5 include Maraviroc Selzentry, Celsentri, UK-427857 (Pfizer), Vicriviroc SCH 417690, SCH-D (Merck), TAK-779 (Takeda), Met-CCL5 Met-RANTES, OTR4120 and OTR4131, Anibamine, DT-13, Aplaviroc (GlaxoSmithKline), GSK706769 (GlaxoSmithKline), INCB009471 (Incyte Corporation) and Cenicriviroc TBR-652, TAK-652 (Takeda).

In order to aid in the understanding and preparation of the within disclosure, the following illustrative, non-limiting, examples are provided, which form part of the description.

### EXAMPLES

### Example 1

### Methods

### Materials & Methods

Clinical assessment strategies. This study was approved by Western University, Human Research Ethics Board (Long-COVID REB ID #120084; COVID-19 REB ID #1670; Healthy Control Subjects REB ID #6963). All patients were screened and enrolled from our tertiary care hospitals (London Health Sciences Centre, London, Ontario, Canada). Both Long-COVID outpatients and acutely ill COVID-19 inpatients had their COVID-19 status confirmed by standard hospital testing for SARS-COV-2 viral genes using polymerase chain reaction (PCR). Long-COVID outpatients were referred to a specialty clinic based on prolonged, diffuse symptoms. Venous blood work was drawn once as part of a larger clinical screen, and subsequent analysis was performed on excess plasma collected for routine blood work by Pathology and Laboratory Medicine (PaLM). COVID-19 inpatients were enrolled on hospital admission on day-1n, either to the medical ward or to the intensive care unit (ICU). Blood from COVID-19 inpatients on day-1 was obtained from indwelling catheters or a venipuncture as required. The healthy control subjects were individuals without disease, acute illness or prescription medications and were previously banked in the Translational Research Centre, London, ON (Directed by Dr. D. D. Fraser; https://translationalresearchcentre.com/). These latter samples were obtained prior to the emergence of SARS-COV-2 in our region and therefore, were considered not to have been exposed to the virus. Final participant groups were matched by age and gender (Long-COVID outpatients with acutely ill COVID-19 inpatients and healthy control subjects) [4]. Blood was centrifuged and plasma was isolated and aliquoted in 250 μL, and frozen at −80° C. until analysis.

Patient demographic and clinical data are shown in Table 4 (Long-COVID outpatients) and in Table 5 (COVID-19 inpatients).

Targeted Proteomics: Proximity Extension Assay (PEA) was used to measure plasma protein expression and included immune-recognition with dNTP-labeled antibodies, extension mediated by polymerases, amplification and detection [30, 35-40]. All clinical samples were analyzed on the same 88 well plate. A control was used to estimate precision, a negative control was used to set background levels and to calculate limit of detection, a plate control to correct levels between plates, and a reference plasma control was used to estimate CV between runs. The relative protein quantification is presented as a Normalized Protein Expression (NPX) on a log 2 scale. Data generation of NPX consists of normalization to the extension control, log 2-transformation, and level adjustment using the plate control. PEA was outsourced to OLINK laboratories (Boston, MA) [39, 41, 42].

### Bioinformatic Analyses

Normalized Protein expression (NPX) data was processed to determine the following: (i) differentially expressed biomarkers, (ii) Gene Ontology (GO) and pathways enrichment, and (ii) affiliation with different cell types. Selected biomarkers were investigated for candidate drugs. The NPX of each peptide was normalized within the specified protein. All normalization computations used the medians to multiply and/or normalize the data. Multiple Mann Whitney Test analysis was performed to determine the differentially expressed proteins. For the functional annotation analysis, data was trained in three steps to be certain that multiple platforms identify similar patterns: (1) analysis with DAVID Bioinformatics Resources (version 6.8; https://david. ncifcrf.gov/) and PANTHER Classification System (version 14.0; http://www.pantherdb.org/), while the protein-protein interaction network analysis was carried out with STRING (Search Tool for the Retrieval of Interacting Genes/Proteins) (version 11.0; https://string-db.org/) [43]; next (2) analysis with Metascape software, a gene annotation and analysis resource (https://metascape.org/gp/index.html #/main/step1) and GSEA, Gene Set Enrichment Analysis platform (https://www.gsea-msigdb.org/gsea/index.jsp); (3) selected biomarkers were analyzed with Kyoto Encyclopedia of Genes and Genomes (KEGG) Mapper software (https://www.genome.jp/kegg/mapper/). Results that converged to the same findings were validated using (i) iPathwayGuide (iPG) (https://www.advaitabio.com) a software program based on KEGG charts (an updated version of KEGG Mapper [44-48] and (ii) Qlucore (https://qlucore.com/geneexpressions). Using iPG, the analysis included the selection of differentially expressed proteins (DEPs) based on a fold change greater than 0.6 (in log 2 scale) as well as a p-value lower than 0.05 after the correction for multiple experiments. The pathways were analyzed with the impact analysis approach [44], which takes into consideration the position of each gene on each pathway, as well as the type and direction of all signals throughout the pathway [44]. Adjusted P values less than 0.05 were considered significant for both GO and pathway analysis.

Immune-cell deconvolution—was completed by CIBERSORT analysis after mapping proteins onto genes (https://cibersort.stanford.edu). CIBERSORT is an analytical free tool provided by Stanford University (“Stanford”) [49].

Matrix visualization was performed by Morpheus software and interacting tools, from Broad Institute (https://software.broadinstitute.org/morpheus). Hierarchical clustering was performed by row, using Pierson one correlation algorithms both for the regular maps and similarity matrixes. K-means analysis was applied for columns to analyze the delineation patterns of the study groups. Principal component analysis was performed with ClustVis software: https://biit.cs.ut.ee/clustvis/.

### Other Statistical Analyses

Bar graphs summarize data analyzed using Mann-Whitney U tests for unpaired data (two-sided). *P<0.05; ***P<0.0001 (GraphPad Prism, version 9). In the analysis process, several public R-studio packages from Bioconductor, were used for the initial global data analysis, as follows: data load (DBI, odbc); data manipulation (tidyr); data visualization (ggplot2, rgl, leaflet); data modeling (tidymodels); data report (shiny); spatial data (maps); web work (xml, github); R writing (devtools, curl).

### Marker Validation

ANGPT1 and VEGFA were measured in human plasma using a custom multiplexed immunoassay kit according to manufacturer's instructions (Endothelial Injury Marker 12-Plex Human ProcartaPlex™ Panel, EPX120-15849-901). Other markers were measured by Enzymelinked Immunosorbent Assay (ELISA). For NK cell marker validation, we used: (a) Human NCAM1 ELISA Kit (CD56) (Abcam: ab119587) and Human CCR7 (Sandwich ELISA) ELISA Kit—(LSBio: LS-F4886). For Neutrophil Extracellular Trap Formation validation, we used EpiQuik Histone H3 Citrullination ELISA Kit (Colorimetric; EpigenTek: P-3095-96) and Human Neutrophil Elastase ELISA Kit (Abcam: ab270204). ELISAs was performed as per the manufacture's protocols, and plasma was diluted 1:20. For validation, we used random patients from a different cohort than those tested by the targeted proteomics (N=11). Plasma samples were tested in sets of three technical replicates.

### Results

Study model and dimensionality reduction of the data sets. FIG. 1A (left panel) illustrates the experimental model consisting of four cohorts, Long-COVID outpatients, acutely ill COVID-19 inpatients (mild and severe) and healthy control subjects. FIG. 1A (right panel) shows the data processing strategy. FIG. 1B presents the principal components analysis (PCA) and hierarchical clustering. Unit variance scaling was applied to rows and SVD with imputation was used to calculate principal components. The X and Y axes show principal component 1 and principal component 2 which explain 67.6% and 21.6% of the total variance, respectively. Data was processed by ClustVis software.

Overall, the data indicated that Long-COVID is a distinct disease compared to COVID-19 ICU patients (severe disease), although the cohorts did share some mechanisms. In contrast, the COVID-19 medical ward inpatients (mild disease) shared mechanisms closer to the healthy control subjects.

Natural killer (NK) cells from Long-COVID changed phenotype from activated to resting. The plasma proteome was deconvoluted to various immune cell-types from tissues based on their complex OMICS profile using the CIBERSORT analysis tool. The proportion of each cell-type in Long-COVID was compared to healthy control subjects (FIG. 2A). Plasma protein patterns suggest that Long-COVID was associated with a shift in NK cells from activated to resting phenotype. The NK phenotype shift was validated by ELISA; CD56 and CCR7 markers were investigated in cohorts containing 11 patients or subjects, which were different from those included in the targeted proteomic groups. While CD56 is a common marker for all NK cells, CCR7 marks the active phenotype and CCR7 was depressed in the Long-COVID outpatients compared to those with acute disease. The NK cell cytotoxic pathways, output of the iPathwaysGuide software, are shown in FIG. 2B. Elevated biomarkers are illustrated with “*” and the down-regulated ones with “**”. Down-regulation of SLP76, an immune response adaptor, was observed while individual markers such as PAK1, MEK1/2 and ERK1/2 were upregulated and interconnected in signaling cascades that target the secretion of TNFα, IFNγ and GM-CSF. Furthermore, the VAV1 exchange factor for Rho-GTP-ases that connects directly to targets like ITGB2 and ITGAM was upregulated, suggesting a subsequent increase in cellular adhesion and movement. FIG. 2C (left panel) shows hierarchical clustering heatmaps, including NK cell signature markers (NCAM/CD56, killer receptors KLRK1 and KLRD1, IFNγ, IL-22, natural cytotoxicity receptor NCR1 and KIT stem cell factor). These markers shared similarity in their behavior based on Pearson correlation algorithms (FIG. 2C, right panel), and they were upregulated in Long-COVID (FIG. 2C, lower panel). FIG. 2D (top left panel) illustrate the pathways analysis scoring with TNFα as the top hit; this graph shows the enrichment p-value on the horizontal axis (in a negative log scale) and the perturbation p-value on the vertical axis [50].

Pathway enrichment analysis demonstrated that TNFα plasma expression was elevated in Long-COVID, as opposed to heathy control subjects (next plot, top row, FIG. 2D). The remaining plots in FIG. 2D portrayed elevated ANGPT1, whereas ANGPT2 did not change suggesting an ANGPT1/ANGPT2 conversion phenomenon, as illustrated by the cartoon (FIG. 2D, bottom, right corner). The ANGPT1/2-Tie axis is critical regulator of endothelial inflammation and vascular leakage [51-53, and excess of ANGPT1 may reduce this process. Potential ‘vascular perturbation’ in Long-COVID pivoting around ANGPT/VEGF axis was validated by ANGPT1 and VEGFA measurement in plasma provided by a different group of patients than those approached by targeted proteomics.

In addition to the NK phenotype shift, the percentual contribution of memory B cells, memory CD4 activated cells and neutrophils to plasma proteome appeared elevated in Long-COVID, whereas resting dendritic cells, CD4 memory resting cells and MI macrophages seemed to be down-regulated (FIG. 2A).

Resetting of immune-cell type proportions is reflected in vascular events mediated by VEGFA. Along with ANGPT1, VEGF signaling is also related to TNFα-linked pathways. The VEGFA pathway could regulate endothelial cell migration. Upregulated markers that are members of the VEGFA pathway in Long-COVID include PXN, SRC, NOS3, and HSPB1. Other key markers such as Flt1 (VEGF receptor 1), AKT and MAPK, indicate endothelial cell survival, migration and proliferation. Key markers capable of protein-protein interactions predict VEGFA induced cell proliferation mediated by SRC and MAP-Kinases, and subsequent AKT signaling to influence cell survival and/or migration.

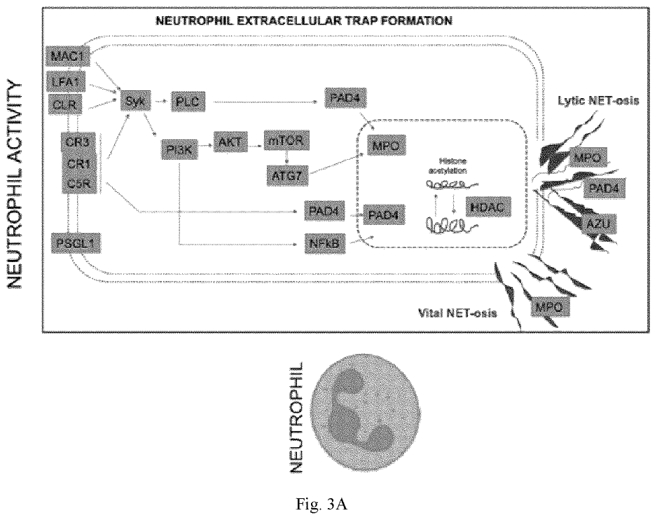

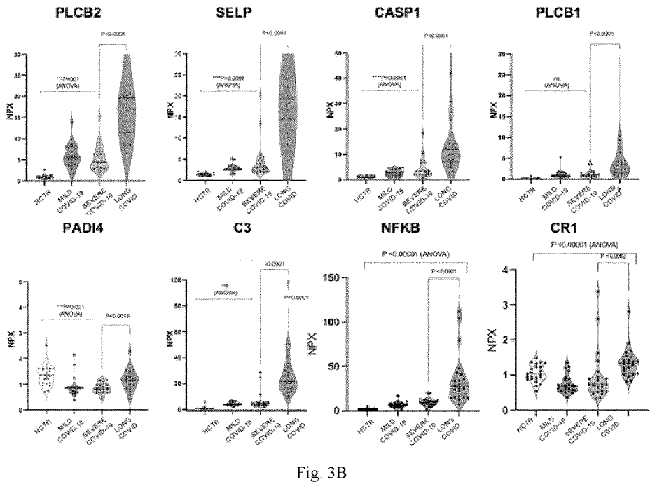

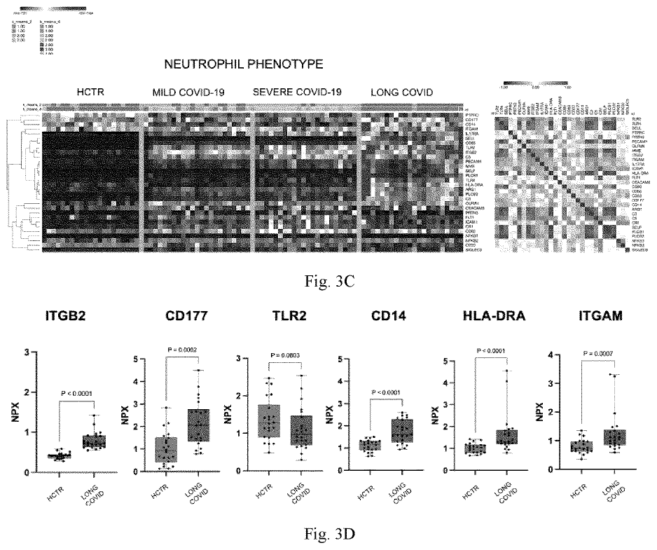

Neutrophil extracellular trap formation in Long-COVID. The features of the neutrophil extracellular traps (NETs) include: i) a defense mechanism against both micro-organisms and sterile triggers, ii) a DNA scaffold with granule-derived proteins, such as enzymatically active proteases and anti-microbial peptides, iii) an important role in inflammation, autoimmunity and other pathophysiological conditions (either detrimental or beneficial), and/or iv) it can be prompted by many triggers and via multiple distinct pathways with often unknown interrelationship [54-57]. The NET pathways shown in FIG. 3A represent a summary of the KEGG NET map output of iPathwayGuide. All boxes in FIG. 3A are overexpressed biomarkers relative to the expression of said biomarkers in healthy controls. Individual NET-specific/related biomarkers were featured in single expression graphs in FIG. 3B, where phospholipase (PLCB2 and PLCB1), CASP1, PDAI4, NFkB, CR1, C3 and SLPG were up-regulated in this cohort when compared to severe COVID-19. While these results appear conflictual, our group and others previously reported a repurposed neutrophil phenotype in severe COVID-19 which may further differentiate in Long-COVID [54-55].

Heatmaps representing the profiling of the neutrophil phenotype (based on a manually curated set of markers taken from specific literature reports) are presented in FIG. 3C [54-57]. The heatmaps show the hierarchical clustering of these markers in healthy control subjects and in COVID-19 patients along with their predicted interdependence (Pearson correlation algorithm; FIG. 3C, right panel). FIG. 3D highlights increased expression of neutrophil CD177, HLA-DRA, ITGAM, ITGB2, and TLR2 manually curated markers in Long-COVID, as compared to other patient cohorts. Interestingly, the presence of CD14, which is an innate immunity marker produced by macrophages and neutrophils was similar in both healthy control subjects and Long-COVID patients, but significantly depleted in mild and severe COVID-19 patients.

NET Inhibitors (anti NET drugs or agents) that can be used in appropriate therapeutic embodiments of the present disclosure include Hydroxychloroquine, Methotrexate, Prednisolone, PAD4, elastase and MPO inhibitors, Rituximab and belimumab, Tocilizumab, Diphenyleneiodonium chloride (DPI) (Molecular and Cellular Biochemistry (2022) 477:673-688).

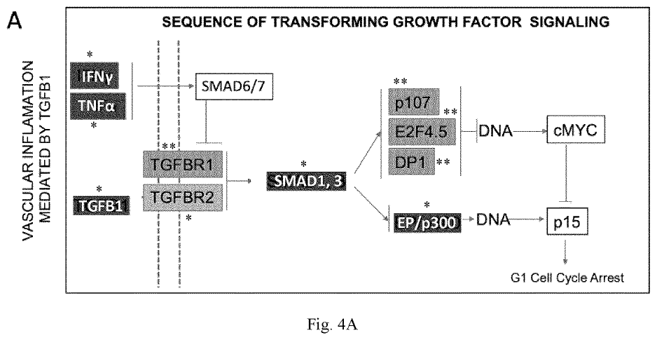

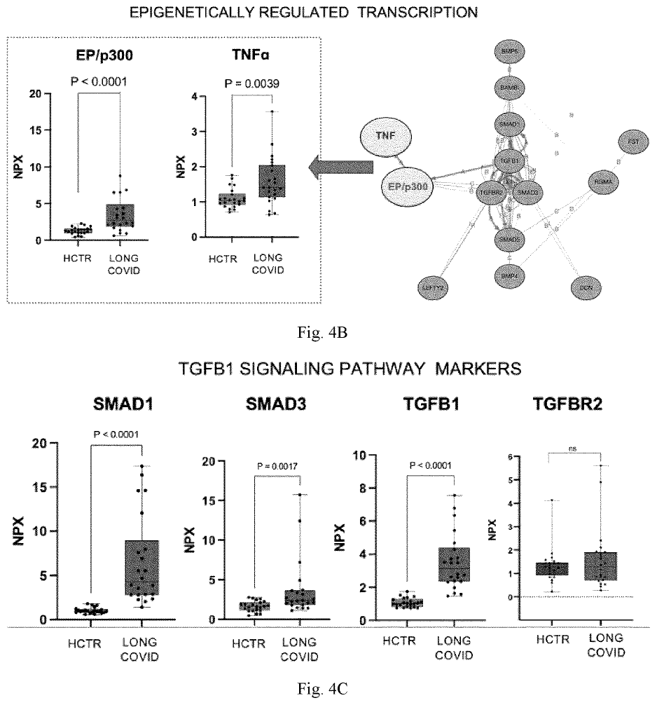

Silent TGFβ signaling and increased EP300 favors vascular inflammation via TNF signaling and subsequent glucocorticoid resistance (GCR). FIG. 4A illustrates TGFβ1 signaling pathway with both up-regulated (*) and down-regulated (**) markers, per KEGG iPathwaysGuide output. FIGS. 4B and 4C graphs represent the expression of the individual markers associated with the TGFβ1 pathway. Key marker interactions are presented in the diagram (FIG. 4B, right). TGFβ1 and TGFBR2 were unchanged from healthy control subjects, thereby favoring TNFα pro-inflammatory signaling to induce an acute form of GCR [56, 58, 59]. TNFα has a significant and broad impact on the transcriptional performance of GR, but no impact on nuclear translocation, dimerization, or DNA binding capacity of GR [59]. The GR cofactor that interacts significantly less with the receptor under GCR conditions is EP/p300, an up-regulated biomarker in Long-COVID that regulates the vascular bed via HIF under hypoxic conditions. EP/p300 strongly influences NFκB activation and thus, mediates inflammation [56]. EP/p300 knockdown reduces the transcriptional output of GCR, whereas its overexpression followed by NFκB inhibition reverts TNFα-induced GCR, trailing an authentic rearrangement model [59]. EP/p300 actions are quite vast, for instance it interacts with SMAD1 to bridge coactivators such as NFκB transcribing IL-6, or it may interact with HIF1α/VEGFA axis [6, 60].

Vascular proliferation via HIF signaling pathway. The Long-COVID proteome has been intersected with the mild and severe COVID-19 data sets, as well as the healthy control group, in a meta-analysis performed by iPathwaysGuide software. The resulted Venn diagram demonstrated that the Long-COVID group shared 80 signaling pathways among groups, 60 with the Severe COVID-19, but still retains 32 unique or independent pathways (FIG. 5A left diagram). Among all the pathways, HIF signaling was a major mechanism that progressed proportionally with disease severity, to a maximum in Long-COVID, as seen in FIG. 5A (where red arrows represent coherent cascades). HIF signaling in mild COVID-19 was initiated by IL-6 and STAT3, and targeted ANGPT and Flt1, followed by possible modifications in angiogenesis, iron metabolism (TFRC effector), vascular tone (HMOX1 effector), and cell survival (through BCL2 survival factor). In severe COVID-19, HIF signaling was triggered by IL-6, IFNγ and growth factors (VEGF and/or EGF) as ligands of receptor tyrosine kinases. Possible downstream modifications include erythropoietin (EPO), angiogenesis (Flt1, EGF, ANGPT1), vascular tone (eNOS, HMOX1), aerobic metabolism (GAPDH, ENO1) and survival (BCL2). HIF activation, and its consequences, in Long-COVID were significantly affected by the simultaneous increase in (EP) p300, predicting extracellular matrix consequences via TIMP1, CD18-dependent inflammation (Integrin β2, ITGB2), and Tie2 regulated angiogenesis. HIF is a leading regulator of hypoxic/ischemic vascular responses, driving transcriptional activation of genes involved in vascular reactivity, angiogenesis, and the deployment of bone marrow-derived angiogenic cells. In parallel, (EP) p300 may function as a histone acetyltransferase with epigenetic functions reflected in endothelial cell proliferation and differentiation [6, 56, 58-61], perhaps contributing to epigenetic modifications induced by SARS-COV-2 infection. (EP) p300 may also affect the stimulation of hypoxia-inducible genes such as VEGF. While there are not therapeutic agents that target (EP) p300, it was predicted by the iPathwayGuide software that the HIF pathway can be however regulated by multiple re-purposed drugs presented in FIG. 5B.

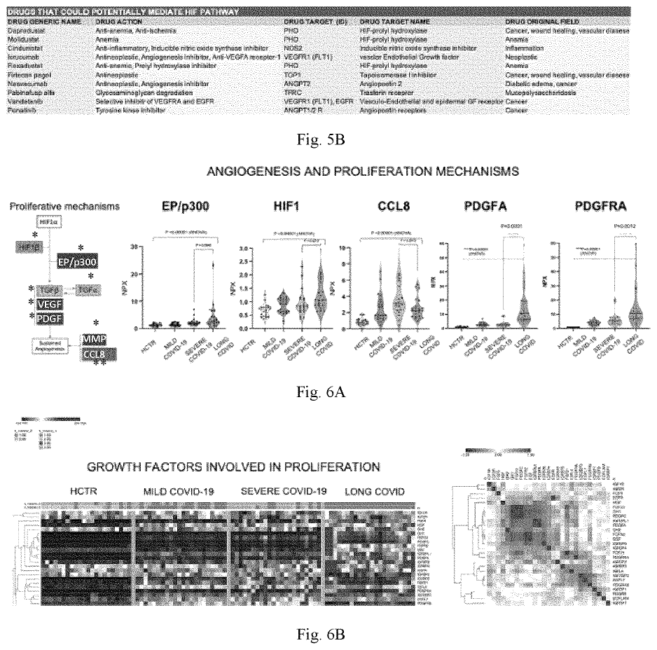

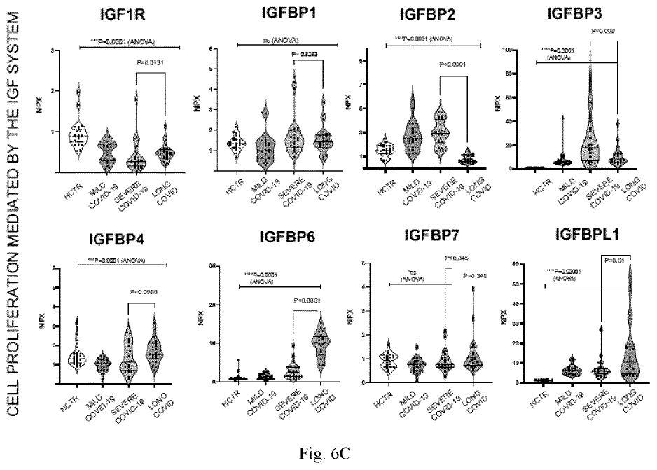

Proliferative pathways in Long-COVID pathology. To establish the nature of the vascular disease and its proliferative aspects, Long-COVID data was investigated with the KEGG cancer pathways, with the most representative signaling cascade led by HIF as highlighted in FIG. 6A (left panel). In this network, the up-regulated biomarkers are shown *and down-regulated * Biomarkers in the up-regulated boxes of FIG. 6A are HIF1β, EP/p300, TGFβ, TGFα, VEGF, PDGF and MMP. The down-regulated biomarker box is CCL8. Plots in FIG. 6A (right panel) illustrate markers elevated in Long-COVID responsible for the sustainability of the vascular bed. Next, FIG. 6B heatmaps reflect the levels of growth-factors that support cell proliferation through RTK membrane receptors located within the vascular bed. Markers have been curated by OLINK. The diagram in the right panel of FIG. 6B illustrates correlations between these factors, in a similarity matrix based on Pearson correlation principles. FIG. 6C graphs show the levels of the insulin-like growth factor binding proteins (IGFBPs) that regulate the bioavailability of insulin-like growth factor (IGF), a factor known to regulate both angiogenesis and sustainability of the vascular bed. Except IGFBP4, the other IGFBPs are common to severe COVID-19. IGFBP6 and IGFBPL1 seem to be Long-COVID specific. The status of the IGFs and their binding proteins (IGFBPs) could reflect the tissue repair capacity.

Long-COVID pathology implies brain dysfunction. The neurologic manifestations of the COVID-19 are well characterized and a comprehensive evaluation of the post-acute neurologic sequelae at 1 year was recently undertaken [62-65]. COVID-19 increased the risk of numerous neurologic sequelae such as ischemic and hemorrhagic stroke, cognition and memory disorders, peripheral nervous system disorders, episodic disorders (migraine and seizures), extrapyramidal and movement disorders, mental health disorders, musculoskeletal disorders, sensory disorders, Guillain-Barré syndrome, encephalitis, and/or encephalopathy. In FIG. 7A, hierarchical clustering heatmaps reflect the levels of neurological markers across the patient groups: healthy controls (HCTR), mild COVID-19, severe COVID-19 and Long-COVID-19 (markers have been curated by OLINK). The values of the PEA expression levels were hierarchically clustered based on Pearson correlation algorithms. Markers selected through the above methodology were investigated for functional annotation using tools from the GSEA platform and MSigDB data repositories (FIG. 7B). This latest analysis demonstrated that functional clusters were formed around leukocyte migration, positive immune signals, glial cell differentiation, neurogenesis and MAPK regulatory modules. Taken together, these pathways predict a possible brain-blood barrier dysfunctionality grounded on cell proliferation. Graphs in FIG. 7C illustrate the expression levels of individual markers from the functional groups presented in FIG. 7B. One of the highly expressed markers, was the amyloid precursor protein (APP) which is known to be a pathognomonic marker for both Alzheimer disease and brain inflammation [62-66]. Additional markers for brain dysfunction include JAM2 (endothelial tight junctions protein), SNAPIN (a mediator of neuronal autophagy-lysosomal function in developing neurons), KCNH2 (potassium channel), S100A14 (involved in cell motility adhesion and growth), KIAA0319 (language impairment biomarker), and IROR1 (a receptor tyrosine kinase like orphan receptor 1, which regulates neurites growth in the central nervous system, mediates WNT-signaling, and maintains the auditive apparatus).

Long-COVID disease implies cardiometabolic injury based on vasculo-proliferative events. COVID-19 is associated with long-term cardiac dysfunction [6, 66]. In FIG. 8A, hierarchical clustering heatmaps reflect the levels and the dynamics of cardio-metabolic markers across cohorts. Markers were curated by OLINK and their expression levels were hierarchically clustered using Morpheus software tools. Topographically, on this map three critical clusters can be observed, and functionally annotated (FIG. 8B) by a standard screening using GSEA/MSigDB tools. This analysis also yielded three functional clusters. Taken in order from 1 to 3, clusters refer to i) extracellular matrix remodeling, ii) cell adhesion and motility and iii) angiogenesis (tube formation). These functional clusters were then investigated for protein-protein interaction (FIG. 8C), where it can be seen that the extracellular matrix remodeling and cell-cell adhesion functions were dominated by integrins (ITGB1, ITGA5, ITGA1, ITGB6, ITGB1B1) which can potentially interact with fibronectin (FN1), filamin (FLNA) and calcium binding markers, essentially mediating Ca²⁺-independent—cell matrix interactions. Graphs in FIG. 8D represent individual marker expression levels in plasma. The integrin changes depicted in these graphs may result into disbalanced extracellular-matrix, leading to apoptosis of the endothelial cells.

To elucidate potential mechanisms in Long-COVID, the plasma proteome of Long-COVID patients was deconvoluted into different cell types and signaling mechanisms, as compared to age- and sex-matched acutely ill COVID-19 inpatients and healthy control subjects. Individual biomarker expression was also analyzed among the patient cohorts to identify potential value in diagnostic and prognostic targets. We found that in Long-COVID patients, natural killer cells switched phenotype from activated to a resting status, and the neutrophils were predisposed to extracellular trap formation. The cell-type proportions and their resetting were directly reflected in vascular events mediated by TNFα, ANGPT1, VEGFA, TGFβ1, and EP300 biomarkers; and the vasculo-proliferative state was anchored by HIF1-pathways. Concurrently, the fluctuations of EP300 may have dramatically influenced HIF1 functions in Long-COVID patients. The “E1A Binding Protein P300 (EP/p300)” is a large protein with multiple cellular functions, including stem cell effector efficacy. Thus, EP/p300 plays a major role in the reprogramming events, leading to a proliferative phenotype (for instance in cancer) with the acquisition of drug resistance and cell plasticity [71]. EP/p300, as a part of various transcriptional complexes, can alter critical biological functions such as cellular proliferation, cell cycle regulation, apoptosis, DNA damage repair, cell fate determination and stem cell pluripotency [71, 72].

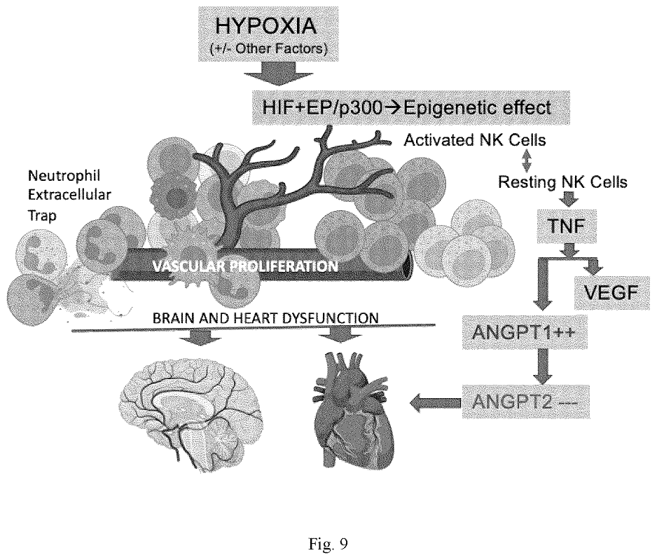

The present disclosure provides insight with regards to Long-COVID pathophysiology and potential preventative strategies (FIG. 9). ANGPT1 has been shown to be up-regulated in Long-COVID [4,5], and this may play an important role in vascular development and angiogenesis, by activating the endothelial cell-specific tyrosine-protein kinase receptor (TEK/Tie receptor) [53]. ANGPT1 may be a critical compensatory protein in Long-COVID, mediating reciprocal interactions between endothelium and the surrounding extracellular matrix (ECM), interacting with the mesenchyme to inhibit endothelial permeability, while sustaining angiogenesis, endothelial cell survival, proliferation, motility and vascular quiescence [53]. In quiescent vessels, ANGPT1 usually forms complexes with TEK kinases from contiguous cells, leading to preferential activation of phosphatidylinositol 3-kinase and the AKT1 signaling cascades. Migrating endothelial cells that lack cell-cell adhesion capacity, rely on ANGT1 to recruit TEK and influence the ECM, resulting in the formation of focal adhesion complexes. However, their interactions with ECM would be impaired in Long-COVID due to down-regulated MMP7, thereby reducing migration and tissue repair. Moreover, depressed MMP7 would impede the proteolytic release of TNF from macrophages, which is typical for hypoxia, and the release of VEGF from its receptor (VEGFR1/Flt1) [73-75]. Elevated ANGPT1 in Long-COVID might be compensatory to the above alterations, thereby contributing to the activation of PTK2/FAK and downstream kinases MAPK1/ERK2 and MAPK3/ERK1; events that ultimately stimulate angiogenesis and blood vessel maturation. Hypoxic conditions occurring in acute COVID-19 would induce growth factors such as VEGF, PDGF, EGF and IGF, ultimately potentiating cell proliferation in the vascular bed, and may hold true for those patients with “happy hypoxia” [76, 77]. Alternatively, ANGPT1 may be a critical link between the VEGFA, HIF and TNFα signaling pathways that were impacted in Long-COVID. VEGFA for instance could play a crucial role in mediating reciprocal interactions between the endothelium and surrounding matrix and mesenchyme, due to the ECM remodeling detected in Long-COVID [4, 6, 53, 56, 58-61].

The present results indicate that Long-COVID can severely impact the functionality of multiple organs via these aforementioned vasculo-proliferative actions. Differential protein expression and network analysis showed disruption of tissue recovery mechanisms that could be directly related to concerted HIF, TNFα and VEGFA signaling actions linked by ANGPT1. Besides providing system-level insights into the mechanism of Long-COVID pathology, our study identified potential biomarkers and therapeutic strategies that might be tailored to specific immune and organ mechanisms to combat the initial effects of hypoxia.

Consistent with our findings, alternatively polarized macrophages were a major contributor to Long-COVID induced molecular alterations [78]. In addition, an anti-inflammatory profile was observed in Long-COVID patients based on insignificant levels of acute phase (IGFγ, IL-1) and macrophage-derived proteins (IL-18, MCP1, sTNFRII) [78]. High expression levels of the APP antigen were measured in plasma from Long-COVID outpatients. The APP gene encodes a cell surface receptor and transmembrane precursor protein that is cleaved by secretases to form a number of peptides, some of which are secreted and can bind to the acetyltransferase complex to promote transcriptional activation, while others form the protein basis of the amyloid plaques found in the brains of patients with Alzheimer disease [62-65]. Mutations in this gene have been implicated in autosomal dominant Alzheimer disease and cerebro-arterial amyloidosis (cerebral amyloid angiopathy). Amyloids were shown to be highly toxic to neuronal cells [62-65]. In this context, and due to the high amounts detected in plasma, our findings suggest that cytotoxic aggregates may be associated with long term neurological symptoms in Long-COVID. Cardiometabolic changes were suggested by in Long-COVID plasma profiles, based on integrin signaling. ITGBP1 and INGA5 seem to engage FN1, to regulate focal adhesion (FAK)-related apoptosis pathways. Elevated CCL5 could maintain an inflammatory state during Long-COVID and together with resting NK cells that express CCR5 [77], promote angiogenesis. The latter via PI3K/AKT, NF-κB, HIF, RAS-ERK-MEK, JAK-STAT and TGFβ-SMAD pathways [78, 79]. However, these mechanisms would be likely independent of the myeloid lines and their inflammatory actions since the myeloid marker MNDA was up-regulated in Long-COVID. Furthermore, the increased levels of HEBP1 suggests a vascular proliferative disease, as this protein includes a natural chemoattractant peptide acting as a natural ligand for formyl peptide receptor-like receptor 2 (FPRL2). The FPRL2 receptor promotes calcium mobilization and chemotaxis in monocytes and dendritic cells, potentially instigating myocarditis [76, 77, 79]. FPRL2 peptides are powerful neutrophil chemotactic factors and activators [76, 77, 79], and possibly instigate neutrophil trap formation. Dysregulated inflammation can potentiate maladaptive healing and pathological remodeling of cardiac tissue, leading to long term cardiac dysfunction, features sometimes present in Long-COVID. As preclinical studies support the use of FPR2 agonists in heart disease, these therapeutic agents may be applicable for the prevention and treatment of cardio-metabolic pathology in Long-COVID [79].

Our study has unveiled potential cell-type signatures and signaling pathways in Long-COVID; however, there are several study limitations worthy of discussion. First, we investigated a limited number of patients, which based on the timing of their infection and data from our regional public health surveillance program, would have been infected with the wild-type or alpha SARS-COV-2 variants. It is currently unknown whether all variants of concern would result in a similar pattern of symptoms and underlying pathophysiology. Second, we deconvoluted plasma proteins with sophisticated software to determine celltype signatures, thereby providing information on cell type contributions to the plasma profiles. Future studies should evaluate cell numbers in fresh blood samples with flowcytometry-based technologies. Third, the plasma proteome was investigated as a surrogate for cell/tissue activities, and when combined with bioinformatics, identified signaling pathways for further investigation. Analyses of the plasma proteome seems reasonable given the broad pathology of this systemic illness, and the practical limitations of obtaining multiple human tissues. Nonetheless, our conclusion must be tempered by the lack of understanding of protein release and turnover. Finally, key signaling pathways were identified, but may still be missing key biomarkers as the proteomics approach used herein was targeted, and not all pathway constituents were available for measurement FIG. 9.

In conclusion, the present disclosure provides a pathophysiological framework to better understand the functional heterogenicity of Long-COVID and provides clues to the neurological and cardio-metabolic basis of this disease.

TABLE 4
Long-COVID Outpatient Demographics and Clinical Data

| | Outpatients |
|---|---|
| Initial Infection Variable | (n = 22) |
| Age (yrs), median (IQR) | 61.0 (20.5) |
| Male sex, no. (%) | 12 (54.5) |
| Diagnostic test: PCR, serology, no. (%) | 22 (100.0) |
| Vaccination status at infection, no. (%) | 2 (9.1) |
| Hospitalization, no. (%) | |
| Ward | 7 (30.4) |
| ICU | 1 (4.3) |
| Comorbidities, no. (%) | |
| Diabetes | 6 (27.3) |
| Hypertension | 8 (36.4) |
| Coronary artery/heart disease | 2 (9.1) |
| Chronic/congestive heart failure | 0 (0.0) |
| Chronic kidney disease | 0 (0.0) |
| Cancer | 1 (4.5) |
| COPD | 0 (0.0) |
| Asthma | 4 (18.2) |
| Presenting symptoms at infection, no. (%) | |
| Fever | 16 (72.7) |
| Cough | 17 (77.3) |
| Anosmia/Ageusia | 13 (59.1) |
| Pharyngitis | 8 (36.4) |
| Headache | 14 (63.6) |
| Confusion/Memory | 2 (9.1) |
| Myalgias | 13 (59.1) |
| Dyspnea | 16 (72.7) |
| Chest pain | 8 (36.4) |
| Nausea/Vomiting/Diarrhea | 11 (50.0) |
| Interventions at infection, no. (%) | |
| Steroids | 6 (27.3) |
| Remdesivir | |
| Tocilizumab | |
| Long-COVID Clinic Variables | |
| Follow up, days from infection onset, median (IQR) | 101.5 (45.5) |
| Lingering symptoms at follow up, no. (%) | |
| Respiratory | 16 (72.7) |
| Cardiovascular | 6 (27.3) |
| Neurology | 8 (36.4) |
| Musculoskeletal | 0 (0.0) |
| Gastro-Intestinal | 3 (13.6) |
| Psychiatric | 1 (4.5) |
| Cutaneous | 0 (0.0) |
| Balance | 0 (0.0) |
| Chest pain | 4 (18.2) |
| Concentration | 0 (0.0) |
| Cough | 2 (9.1) |
| Dyspnea | 16 (72.7) |
| Fatigue | 11 (50.0) |
| Headache | 2 (9.1) |
| Low mood | 1 (4.5) |
| Anxiety | 1 (4.5) |
| Memory | 6 (27.3) |
| Nausea | 1 (4.5) |
| Palpitations | 1 (4.5) |
| Paresthesia | 1 (4.5) |
| Smell/taste | 2 (9.1) |
| Word finding | 1 (4.5) |
| Non-specific | 11 (50.0) |
| Laboratories at follow up, median (IQR) | |
| White blood cell count | 7.1 (1.9) |
| Neutrophils | 4.5 (1.5) |
| Lymphocytes | 2.0 (0.7) |
| Hemoglobin | 139.5 (24.8) |
| Platelets | 239.5 (64.2) |
| C-Reactive Protein (CRP) | 1.8 (3.5) |
| Ferritin | 76.0 (118.8) |
| Lactate Dehydrogenase (LDH) | 206.0 (39.0) |
| Alanine Aminotransferase (ALT) | 20.0 (11.2) |
| Interventions at follow up, no. (%) | |
| Pulmicort | 1 (4.5) |
| Anticoagulant | 1 (4.5) |
| Symbicort | 10 (45.5) |
| Ventolin | 3 (13.6) |
| Lasix | 1 (4.5) |
| Nasal spray | 2 (9.1) |
| Oxygen | 2 (9.1) |
| Physiotherapy | 4 (18.2) |
| None | 8 (36.4) |

TABLE 5
Acutely Ill COVID-19 Inpatient Demographics and Clinical Data

| | Ward | ICU |
|---|---|---|
| | Inpatients | Inpatients |
| Variable | (n = 22) | (n = 22) |
| Age (yrs), median (IQR) | 60.0 (21.5) | 58.0 (17.5) |
| Male sex, no. (%) | 12 (54.5) | 12 (54.5) |
| Weight (kg), median (IQR) | 84.8 (14.8) | 90.0 (28.3) |
| Height (cm), median (IQR) | 169.0 (9.2) | 170.0 (9.0) |
| BMI, median (IQR) | 28.6 (5.6) | 30.5 (7.6) |
| MODS, median (IQR) | | 5.0 (1.0) |
| SOFA Score, median (IQR) | | 5.5 (5.8) |
| Comorbidities, no. (%) | | |
| Diabetes | 4 (18.2) | 10 (45.5) |
| Hypertension | 9 (40.9) | 9 (40.9) |
| Coronary artery/heart disease | 1 (4.5) | 2 (9.1) |
| Chronic/congestive heart failure | 0 (0.0) | 0 (0.0) |
| Chronic kidney disease | 1 (4.5) | 2 (9.1) |
| Cancer | 3 (13.6) | 2 (9.1) |
| COPD | 0 (0.0) | 1 (4.5) |
| Presenting symptoms, no. (%) | | |
| Fever | 18 (81.8) | — |
| Cough | 18 (81.8) | — |
| Anosmia/Ageusia | 4 (18.2) | — |
| Pharyngitis | 4 (18.2) | — |
| Headache | 3 (13.6) | — |
| Myalgias | 14 (63.6) | — |
| Dyspnea | 20 (90.9) | — |
| Chest pain | 3 (13.6) | — |
| Nausea/Vomiting/Diarrhea | 9 (40.9) | — |
| Pulmonary pathology, no. (%) | | |
| Unilateral pneumonia | — | 1 (4.5) |
| Bilateral pneumonia | 21 (95.5) | 20 (90.9) |
| Interstitial infiltrates/R effusion | — | 1 (4.5) |
| Laboratories, median (IQR) | | |
| Hemoglobin | 129.5 (23.0) | 118.5 (29.8) |
| White Blood Cell count | 6.8 (4.9) | 8.8 (7.9) |
| Neutrophils | 5.8 (3.9) | 7.5 (7.4) |
| Lymphocytes | 0.8 (0.7) | 0.7 (0.6) |
| Platelets | 210.0 (68.5) | 220.0 (143.5) |
| Creatinine | 69.5 (25.5) | 79.5 (86.2) |
| International Normalized Ratio | 1.0 (0.1) | 1.2 (0.1) |
| Lactate | 1.7 (0.9) | 1.2 (0.8) |
| Partial thromboplastin time (PTT) | — | 26.5 (5.0) |
| PaO₂/FiO₂ Ratio | — | 128.5 (62.5) |
| Interventions, no. (%) | | |
| Renal replacement therapy | 0 (0.0) | 5 (22.7) |
| High-flow nasal cannula | 13 (59.1) | 15 (68.2) |
| Non-invasive mechanical ventilation | 1 (4.5) | 6 (27.3) |
| Invasive mechanical ventilation | 2 (9.1) | 20 (90.9) |
| Extracorporeal membrane | 0 (0.0) | 1 (4.5) |
| oxygenation | | |
| Tocilizumab | 2 (9.1) | 0 (0.0) |
| Steroids | 21 (95.5) | 14 (63.6) |
| Vasoactive medications | 2 (10.0) | 18 (81.8) |
| Antibiotics | 22 (100.0) | 22 (100.0) |
| Anti-virals | 4 (18.2) | 3 (13.6) |
| Antiplatelet | 4 (18.2) | 17 (77.3) |
| Anticoagulation | 22 (100.0) | 21 (95.5) |
| Outcomes | | |
| Days, median (IQR) | 9.0 (6.8) | 15.5 (15.0) |
| Died, no. (%) | 2 (9.1) | 10 (45.5) |

### REFERENCES

1. Castanares-Zapatero D, Chalon P, Kohn L, Dauvrin M, Detollenaere J, et al. Pathophysiology and mechanism of Long-COVID: a comprehensive review. Ann Med. 2022; 54 (1): 1473-87.
2. Brown K, Yahyouche A, Haroon S, Camaradou J, Turner J. Long-COVID and self-management. Lancet. 2022; 22 (399): 10322-55.
3. World Health Organization WHO R&D Blueprint. Novel Coronavirus. COVID-19 Therapeutic Trial Synopsis.https://cdn.who.int/media/docs/default-source/blue-print/covid-19-therapeutic-trial-synopsis.pdf?sfvrsn=44b83 344_1&download=true.
4. Patel M A, Knauer M J, Nicholson M, Daley M, Van Nynatten L R, Martin C, et al. Elevated vascular transformation blood biomarkers in long-COVID indicate angiogenesis as a key pathophysiological mechanism. Molec Med. 2022; 28 (1): 122. https://doi.org/10.1186/s10020-022-00548-8.
5. Patel M A, Knauer M J, Nicholson M, Daley M, Van Nynatten L R, Cepinskas G, Fraser D D. Organ and cell-specific biomarkers of Long-COVID identified with targeted proteomics and machine learning. Mol Med. 2023; 29 (1): 26.
6. Al-Aly Z, Xie Y, Bowe B. High-dimensional characterization of post-acute sequelae of COVID-19. Nature. 2021; 594 (7862): 259-64.
7. Yong S J, Shiliang L. Proposed subtypes of post-COVID-19 syndrome (or long-COVID) and their respective potential therapies. Rev Med Virol. 2022; 32 (4): e2315.
8. Antonelli M, Pujol J C, Spector T D, Ourselin S, Steves C J. Risk of Long-COVID associated with delta versus omicron variants of SARS-COV-2. Lancet. 2022; 399 (10343): 2263-4.
9. Couzin-Frankel J, Vogel G. Vaccines may cause rare, long-COVID-like symptoms. Science. 2022; 375 (6579): 364-6.
10. Dotan A, David P, Arnheim D, Shoenfeld Y. The autonomic aspects of the post-COVID19 syndrome. Autoimmun Rev. 2022; 21 (5): 103071.
11. Ledford H. Long-COVID treatments: why the world is still waiting. Nature. 2022; 608 (7922): 258-60.
12. Su Y, Yuan D, Chen D G, Ng R H, Wang K, Choi J, Li S, Hong S, Zhang R, Xie J, Kornilov S A, Scherler K, et al. Multiple early factors anticipate post-acute COVID-19 sequelae. Cell. 2022; 185 (5): 881-95.
13. Vijayakumar B, Boustani K, Ogger P P, Papadaki A, Tonkin J, Orton C M, Ghai P, Suveizdyte K, et al. Immuno-proteomic profiling reveals aberrant immune cell regulation in the airways of individuals with ongoing post-COVID-19 respiratory disease. Immunity. 2022; 55 (3): 542-56.
14. Fraser D D, Cepinskas G, Slessarev M, Martin C, Daley M, Miller M R, et al. Inflammation Profiling of Critically Ill Coronavirus Disease 2019 Patients. Crit Care Explor. 2020; 2 (6): e0144. https://doi.org/10.1097/CCE.0000000000000144
15. Dong E, Du H, Gardner H. An interactive web-based dashboard to track COVID-19 in real time. Lancet Infect Dis. 2020; 20:533-4.
16. Nalbandian A, Sehgal K, Gupta A, Madhavan M V, McGroder C, Stevens J S, Cook J R, Nordvig A S, Shalev D, et al. Post-acute COVID-19 syndrome. Nat Med Nat Med. 2021; 27 (4): 601-15.
17. Taribagil P, Creer D, Tahir D. ‘Long-COVID’ syndrome. BMJ Case Rep. 2021; 14: e241485. https://doi.org/10.1136/bcr-2020-241485.
18. Al-Aly Z, Bowe B, Xie Y. Long-COVID after breakthrough SARS-COV-2 infection. Nat Med. 2022; 28 (7): 1461-7.
19. Wilk A J, Rustagi A, Zhao N Q, Roque J, Martínez-Colón G J, McKechnie J L, et al. A single-cell atlas of the peripheral immune response in patients with severe COVID-19. Nat Med. 2020; 26:1070-6.
20. Kruger A, Vlok M, Turner S, Venter C, Laubscher G J, Kell D B, Pretorius E. Proteomics of fibrin amyloid microclots in Long-COVID/post-acute sequelae of COVID-19 (PASC) shows many entrapped pro-inflammatory molecules that may also contribute to a failed fibrinolytic system. Cardiovasc Diabeto. 2022; 21 (1): 190. https://doi.org/10.1186/s12933-022-01623-4.
21. Demichev V, Tober-Lau P, Lemke O, Nazarenko T, Thibeault C, Whitwell H, Röhl A, Freiwald A, Szyrwiel L, Ludwig D, Correia-Melo C, et al. A time-resolved proteomic and prognostic map of COVID-19. Cell Syst. 2021; 12 (8): 780-94.
22. Filbin M R, et al. Longitudinal proteomic analysis of severe COVID-19 reveals survival-associated signatures, tissue-specific cell death, and cell-cell interactions. Cell Rep Med. 2021; 2 (5): 100287.
23. Filbin M R, Mehta A, Schneider A M, Kays K R, Guess J R, Gentili M, Fenyves B G, Charland N C, Gonye A L K, et al. Novel outcome biomarkers identified with targeted proteomic analyses of plasma from critically Ill coronavirus disease 2019 patients. Cell Rep Med. 2021; 2 (5): 100287.
24. Memon D, Barrio-Hernandez I, Beltrao P. Individual COVID-19 disease trajectories revealed by plasma proteomics. EMBO Mol Med. 2021; 13 (8): e14532.
25. Vedula P, Tang H Y, Speicher D W, Kashina A. Protein posttranslational signatures identified in COVID-19 patient plasma. Front Cell Dev Biol. 2022; 10:807149.
26. Al-Nesf M A Y, Abdesselem H B, Bensmail I, Ibrahim S, Saeed W A H, Mohammed S S I, Razok A, Alhussain H, et al. Prognostic tools and candidate drugs based on plasma proteomics of patients with severe COVID-19 complications. Nat Commun. 2022; 13 (1): 946. https://doi.org/10.1038/s41467-022-28639-4.
27. Feyaerts D, Hédou J, Gillard J, Chen H, Tsai E S, Peterson L S, et al. Integrated plasma proteomic and single-cell immune signaling network signatures demarcate mild, moderate, and severe COVID-19. bioRxiv. 2021. https://doi.org/0.1016/j.xcrm.2022.100680.
28. Lam S M, Zhang C, Wang Z, Ni Z, Zhang S, Yang S, Huang X, Mo L, et al. A multi-omics investigation of the composition and function of extracellular vesicles along the temporal trajectory of COVID-19. Nat Metab. 2021; 3 (7): 909-22.
29. Rostron A J, Simpson A J, Hambleton S, Laurenti E, Lyons P A, Meyer K B, Nikolić M Z, Duncan C J A, Smith K G C, et al. Single-cell multi-omics analysis of the immune response in COVID-19. Nat Med. 2021; 27 (5): 904-16.
30. Iosef C, Martin C M, Slessarev M, Gillio-Meina C, Cepinskas G, Han V K M, Fraser D D. COVID-19 plasma proteome reveals novel temporal and cell-specific signatures for disease severity and high-precision disease management. J Cell Mol Med. 2023; 27 (1): 141-57. https://doi. org/10.1111/jcmm.17622.
31. Mathew D, Giles J R, Baxter A E, Oldridge D A, Greenplate A R, Wu J E, Alanio C, Kuri-Cervantes L, Pampena M B, D′Andrea K, Manne S, et al. Deep immune profiling of COVID-19 patients reveals distinct immunotypes with therapeutic implications. Science. 2020; 369 (6508): 8511.
32. Laing A G, Lorenc A, Del Molino Barrio I, Das A, Fish M, Monin L, Muñoz-Ruiz M, Mckenzie D R, Hayday T S, Francos-Quijorna I, Kamdar S, et al. A dynamic COVID-19 immune signature includes associations with poor prognosis. Nat Med. 2020; 26:1623-35.
33. Fraser D D, Cepinskas G, Slessarev M, Martin C M, Daley M, Patel M A, et al. Detection and profiling of human coronavirus immunoglobulins in critically Ill coronavirus disease 2019 patients. Crit Care Explor. 2021; 3 (3): e0369. https://doi.org/10.1097/CCE.0000000000000369.
34. Lupisella L A, Parvin S S, Wurtz T R, Garcia R A. Formyl peptide receptor 2 and heart disease. Semin Immunol. 2022. https://doi.org/10.1016/j.smim.2022. 101602.
35. Lundberg M, Eriksson A, Tran B, Assarsson E, Fredrickson S. Homogenous antibody-based proximity ligation assay provides sensitive and specific detection or low abundant proteins in human blood. Nucl Acid Res. 2011; 39 (15): e102. https://doi.org/10.1093/nar/gkr424.
36. Arunachalam P S, Wimmers F, Mok C K P, Perera R A P M, Scott M, Hagan T, Sigal N, Feng Y, Bristow L, et al. Systems biological assessment of immunity to mild versus severe COVID-19 infection in humans. Science. 2020; 369 (6508): 1210-20. https://doi.org/10.1126/science.abc6261.
37. Møller P L, Rohde P D, Winther S, Breining P, Nissen L, Nykjaer A, Bøttcher M, Nyegaard M, Kjolby M. Sortilin as a biomarker for cardiovascular disease revisited. Front Cardiovasc Med. 2021; 8:652584. https://doi.org/10.3389/fcvm.2021.652584.
38. Zhao J, Schank M, Wang L, Dang X, Cao D, Khanal S, Nguyen LNT, Zhang Y, Wu X Y, Adkins J L, Pelton B J, et al. Plasma biomarkers for systemic inflammation in COVID-19 survivors. Proteomics Clin Appl. 2022; 16 (5): e2200031. https://doi.org/10.1002/prca.20220 0031.
39. Shu T, Ning W, Wu D, Xu J, Han Q, Huang M, Zou X, Yang Q, Yuan Y, Bie Y, Pan S, Mu J, Han Y, et al. Plasma proteomics identify biomarkers and pathogenesis of COVID-19. Immunity. 2020; 53 (5): 1108-22. https://doi.org/10.1016/j.immuni.2020.10.008.
40. Fraser D D, Cepinskas G, Patterson E K, Slessarev M, Martin C, Daley M, Patel M A, Miller M R, O'Gorman D B, Gill S E, Pare G, Prassas I, Diamandis E. Novel Outcome Biomarkers Identified With Targeted Proteomic Analyses of Plasma From Critically Ill Coronavirus Disease 2019 Patients. Crit Care Explor. 2020; 2 (9): e0189. https://doi. org/10.1097/CCE. 00000 00000 000189.
41. Williams S A, Kivimaki M, Langenberg C, Hingorani A D, Casas J P, Bouchard C, Jonasson C, Sarzynski M A, et al. Plasma protein patterns as comprehensive indicators of health. Nat Med. 2019; 25 (12): 1851-7.
42. Szklarczyk D, Gable A L, Lyon D, Junge A, Wyder S, Huerta-Cepas J, Simonovic M, Doncheva N T, Morris J H, Bork P, Jensen L J, Mering C V. STRING v11: protein-protein association networks with increased coverage, supporting functional discovery in genome-wide experimental datasets. Nucleic Acids Res. 2019; 47 (D1): D607-13. https://doi.org/10.1093/nar/gkyl1 31.
43. Draghici S, Khatri P, Tarca A L, Amin K, Done A, Voichita C, Georgescu C, Romero R. A systems biology approach for pathway level analysis. Genome Res. 2007; 17 (10): 1537-45.
44. Sun B B, Maranville J C, Peters J E, Stacey D, Staley J R, Blackshaw J, Burgess S, Jiang T, Paige E, et al. Genomic atlas of the human plasma proteome. Nature. 2018; 558 (7708): 73-9. https://doi.org/10.1038/s41586-018-0175-2.
45. Shafi A, Nguyen T, Peyvandipour A, Nguyen H, Draghici S. A multi-cohort and multi-omics meta-analysis framework to identify network-based gene signatures. Front Genet. 2019; 19 (10): 159. https://doi. org/10.3389/fgene.2019.00159.
46. Nguyen T, Shafi A, Nguyen T M, Schissler A G, Draghici S. N B I A: a networkbased integrative analysis framework—applied to pathway analysis. Sci Rep. 2020; 10 (1): 4188. https://doi.org/10.1038/s41598-020-60981-9.
47. Pfaff E R, Girvin A T, Bennett T D, Bhatia A, Brooks I M, Deer R R, Dekermanjian J P, Jolley S E, Kahn M G, Kostka K, et al. N3C Consortium Identifying who has long COVID in the USA a machine learning approach using N3C data. Lancet Digit Health. 2022. https://doi.org/10.1016/S2589-7500 (22) 00048-6.
48. Newman A M, Liu C L, Green M R, Gentles A J, Feng W, Xu Y, Hoang C D, Diehn M, Alizadeh A A. Robust enumeration of cell subsets from tissue expression profiles. Nat Methods. 2015; 12 (5): 453-7. https://doi.org/10.1038/nmeth.3337.
49. Mathew D, Giles J R, Baxter A E, Oldridge D A, Greenplate A R, Wu J E, Alanio C, Kuri-Cervantes L, Pampena M B, D′Andrea K, Manne S, et al. Deep immune profiling of COVID-19 patients reveals distinct immunotypes with therapeutic implications. Science. 2020; 369 (6508): 8511. https://doi.org/10.1126/science.abc8511.
50. Laing A G, Lorenc A, Molino Del, Del Barrio I, Das A, Fish M, Monin L, Muñoz-Ruiz M, Mckenzie D R, Hayday T S, et al. A dynamic COVID-19 immune signature includes associations with poor prognosis. Nat Med. 2020; 26:1623-35.
51. Parikh S M. The angiopoietins and Tie in vascular inflammation. Curr Oppinion Hematol. 2017; 24 (5): 432-8.
52. Silvin A, Chapuis N, Dunsmore G, Goubet A G, Dubuisson A, Derosa L, Almire C, Hénon C, Kosmider O, Droin N, Rameau P, Catelain C, Alfaro A, et al. Elevated calprotection and abnormal myeloid cell subsets discriminate severe from mild COVID19. Cell. 2020; 182:1401-18.
53. Rice C M, Lewis P, Ponce-Garcia F M, Willem Gibbs D, Cela F, et al. Neutrophils in COVID19 are characterized by hyperactive immature state and maintained CXR2 expression. medRix. 2022. https://doi.org/10.1101/2022.03.23.22272828.
54. Sinha S, Rosin N L, Arora R, Labit E, Jaffer A, Cao L, Farias R, Nguyen A P, de Almeida L G N, et al. Dexamethasone modulates immature neutrophils and interferon programming in severe COVID-19. Nat Med. 2022; 28 (1): 201-11.
55. Meizlish M L, Pine A B, Bishai J D, Goshua G, Nadelmann E R, Simonov M, Chang C H, Zhang H, Shallow M, Bahel P, Owusu K, et al. A neutrophil activation signature predicts critical illness and mortality in COVID-19. Blood Adv. 2021; 5 (5): 1164-77.
56. Liu Z W, Zhang Y M, Zhang L Y, Zhou T, Li Y Y, Zhou G C, Miao Z M, et al. Duality of interactions between TGF-β and TNF-α during tumor formation. Front Immunol. 2021. https://doi.org/10.3389/fimmu.12:810286.10.3389/fimmu.2021.810286.
57. Portuguez Aj, Grbesa I, Tal M, Deitch R, Raz D, Kliker L, Weismann R, Schwartz M, Loza O, Cohen L, Marchenkov-Flam L, et al. Ep300 sequestration to functionally distinct glucocorticoid receptor binding loci underlie rapid gene activation and repression. Nucl Ac Res. 2022; 50 (12): 6702-14.
58. Dendoncker K, Timmermans S, Vandewalle J, Eggermont M, Lempiäinen J, Paakinaho V, Van Hamme E, Dewaele S, Vandevyver S, et al. TNF-α inhibits glucocorticoid receptor-induced gene expression by reshaping the GR nuclear cofactor profile. Proc Natl Acad Sci USA. 2019; 116 (26): 12942-51.
59. Morikawa, et al. Genome-wide mechanisms of Smad binding. Oncogene. 2013; 32:1609-15.
60. Hugon J, Msika E F, Queneau M, Farid K, Paquet C. Long-COVID: cognitive complaints (brain fog) and dysfunction of the cingulate cortex. J Neurol. 2022; 269 (1): 44-6. https://doi.org/0.1007/s00415-021-10655-x.
61. ENTREZ: https://www.ncbi.nlm.nih.gov/gene/4088; https://www.ncbi.nlm.nih.gov/gene? Db=gene&Cmd=ShowDetailView&TermToSearch=2033
62. Boldrini M, Canoll P D, Klein R S. How COVID-19 affects the brain. JAMA Psychiat. 2021. https://doi.org/10.1001/jamapsychiatry.2021.0500.
63. Ellul M A, Benjamin L, Singh B, Lant S, Michael B D, Easton A, Kneen R, Defres S, Sejvar J, Solomon T. Neurological associations of COVID-19. Lancet Neurol. 2020; 19:767-83. https://doi. org/10.1016/S1474-4422 (20) 30221-0.
64. Charnley M, Islam S, Bindra G K, Engwirda J, Ratcliffe J, Zhou J, Mezzenga R, Hulett M D, Han K, Berryman J T, Reynolds N P. Neurotoxic amyloidogenic peptides in the proteome of SARS-COV2: potential implications for neurological symptoms in COVID-19. Nat Commun. 2022; 13 (1): 3387. https://doi.org/10.1038/s41467-022-30932-1.
65. Asaduzzaman M, Constantinou S, Min H, Gallon J, Lin M L, Singh P, Raguz S, Ali S, Shousha S, Coombes R C, Lam E W, Hu Y, Yagüe E. Tumour suppressor EP300, a modulator of paclitaxel resistance and stemness, is downregulated in metaplastic breast cancer. Breast Cancer Res Treat. 2018; 167 (2): 605-6.
66. Xie Y, Xu E, Bowe B, Al-Aly Z. Long-term cardiovascular outcomes of COVID-19. Nat Med. 2022; 28:583-90. https://doi.org/10.1038/s41591-022-01689-3.
67. Zeng Z, Lan T, Wei Y, Wei X. CCL5/CCR5 axis in human diseases and related treatments. Genes Dis. 2022; 9 (1): 12-27.
68. Ring A, Kaur P, Lang J E. EP300 knockdown reduces cancer stem cell phenotype, tumor growth and metastasis in triple negative breast cancer. BMC Cancer. 2020; 20 (1): 1076.
69. Pesce S, Moretta L, Moretta A, Marcenaro E. Human NK cell subsets redistribution in pathological conditions: a role for CCR7 receptor. Front Immunol. 2016; 7 (7): 414.
70. Ito T K, Ishi G, Saito S, Iano K, Suziki T, Ochiai A, Ito T K, Ishi G, Saito S, Iano K, Suziki T, Ochiai A. Degradation of soluble VEGF receptor-1 by MMP-7 allows VEGF access to endothelial cells. Blood. 2009; 113 (10): 2363-9.
71. Patterson B K, Guevara-Coto J, Yogendra R, Francisco E B, Long E, Pise A, Rodrigues H, Parikh P, Mora J, Mora-Rodríguez R A. Immune-based prediction of COVID-19 severity and chronicity decoded using machine learning. Front Immunol. 2021; 28 (12): 700782.
72. Patterson B K, Francisco E B, Yogendra R, Long E, Pise A, Rodrigues H, Hall E, et al. Persistence of SARS COV-2 S1 protein in CD16+ monocytes in postacute sequelae of COVID-19 (PASC) up to 15 months post-infection. Front Immunol. 2022; 10 (12): 746021.
73. Dhont S, Derom E, Van Braeckel E, Depuydt P, Lambrecht B N. The pathophysiology of ‘happy’ hypoxemia in COVID-19. Respir Res. 2020; 21:198.
74. Akoumianaki E, Vaporidi K, Bolaki M, Georgopoulos D. Happy or silent hypoxia in COVID-19—a misnomer born in the pandemic era. Front Physiol. 2021; 12:745634.
75. Robertson M. Role of chemokines in the biology of natural killer cells. J Leukoc Biol. 2002; 71 (2): 173-83.
76. Zeng Z, Lan T, Wei Y, Wei X. CCL5/CCR5 axis in human diseases and related treatments. Genes Dis. 2022; 9 (1): 12-27.
77. Kovarik J J, Bileck A, Hagn G, Meier-Menches S M, Frey T, Kaempf A, Hollenstein M, Shoumariyeh T, Skos L, Reiter B, Gerner M C, Spannbauer A, Hasimbegovic E, Schmidl D, Garhöfer G, Gyöngyösi M, Schmetterer K G, Gerner C. A multi-omics based anti-inflammatory immune signature characterizes long COVID-19 syndrome. iScience. 2023; 26 (1): 105717.
78. Witkowski M, Tizian C, Ferreira-Gomes M, et al. Untimely TGFβ responses in COVID-19 limit antiviral functions of NK cells. Nature. 2021; 600:295-301.
79. Adang E A M C, Strous M T A, van den Bergh J P, Gach D, van Kampen V E M, van Zeeland R E P, Barten D G, van Osch F H M. Association of heart rate variability with pulmonary function impairment and symptomatology post-COVID-19 hospitalization. Sensors. 2023; 23 (5): 2473.

Through the embodiments that are illustrated and described, the currently contemplated best mode of making and using the disclosure is described. Without further elaboration, it is believed that one of ordinary skill in the art can, based on the description presented herein, utilize the present disclosure to the full extent. All publications cited herein are incorporated by reference.

Although the description above contains many specificities, these should not be construed as limiting the scope of the disclosure, but as merely providing illustrations of some of the presently embodiments of this disclosure.

1. A method of determining a risk of developing a neurological disorder in a Long-COVID patient comprising: (a) testing levels of at least one marker associated with a neurologic disorder in a blood plasma sample taken from the Long-COVID patient, and (b) making a determination using machine learning that the Long-COVID patient is at risk of developing said neurological disorder when the levels of said at least one marker is increased in the Long-COVID patient compared to healthy control reference levels of said at least one marker, the at least one marker associated with the neurological disorder includes KIAA0319, S100A14, SNAPIN, IL6, SOX2, FCER2, APP, HAVCR2, HP1R, JAM2, IL34, ADGRB3, KCNH2, GBA, GHRHR, DSCAM, EPH86, DUSP29, PIK3IPI, IGFBP4 and CACNB3.

2. The method of claim 1, wherein the at least one marker associated with the neurologic disorder includes KIAA0319, S100A14 and SNAPIN.

3. (canceled)

4. The method of claim 1, wherein the method further comprises treating the Long-COVID patient with a drug or agent effective to prevent or treat the neurological disorder.

5-14. (canceled)

15. A method of treating a Long-COVID patient, the method comprising (a) providing a Long-COVID patient having an increased level of expression of at least one marker associated with a neurologic disorder relative to a healthy subject, and (b) treating the Long-COVID patient with a drug or agent effective to reduce the level of expression of the at least one marker associated with the neurologic disorder, wherein the at least one marker associated with the neurologic disorder is at least one of S100A14, KIAA0319, SNAPIN, APP, JAM2, KCNH2, and IROR1.

16. (canceled)

17. The method of claim 15, wherein the marker is APP the neurological disorder is Alzheimer's disease, and the drug includes at least one drug or agent effective to prevent or treat Alzheimer's disease.

18. The method of claim 17, wherein the at least one drug or agent effective to prevent or treat Alzheimer's disease include Aducanumab, Gantenerumab, BAN2401, ALZ-801, Lecanemab, Bepranemab, Galantamine, rivastigmine and donepezil.

19. The method of claim 16, wherein the at least one marker is KCNH2, and the drug or agent is a KCNH2 inhibitor.

20-27. (canceled)

28. A method of determining a risk of developing a neurological disorder in a Long-COVID patient comprising: (a) testing levels of at least one marker associated with a neurologic disorder in a blood plasma sample taken from the Long-COVID patient, and (b) making a determination that the Long-COVID patient is at risk of developing said neurological disorder when the levels of said at least one marker is increased in the Long-COVID patient compared to healthy control reference levels of said at least one marker, the at least one marker associated with the neurological disorder includes KIAA0319, S100A14, SNAPIN, IL6, SOX2, FCER2, APP, HAVCR2, HP1R, JAM2, IL34, ADGRB3, KCNH2, GBA, GHRHR, DSCAM, EPH86, DUSP29, PIK3IPI, IGFBP4 and CACNB3, wherein the method further comprises treating the Long-COVID patient with a drug or agent effective to prevent or treat the neurological disorder.

29. The method of claim 28, wherein the at least one marker associated with the neurologic disorder includes KIAA0319, S100A14 and SNAPIN.

30. The method of claim 28, wherein said drug or agent is effective to reduce the level of expression of the at least one marker associated with the neurologic disorder.
